(19) United States
(12) Patent Application Publication
(10) Pub. No.: US 2004/0058385 A1
(43) Pub. Date: Mar. 25, 2004

(54) Kit and method for determining multiple analytes, with provisions for refrencing the density of immobilised recognition elements

(76) Inventors: Andreas Peter Abel, Basel (CH); Gerhard Matthias Kresbach, Staufen (DE); Gert Ludwig Duveneck, Bad Krozingen (DE); Nania Graciela Scharer-Hernandez, Gelterkinden (CH)

Correspondence Address: WENDEROTH, LIND & PONACK, L.L.P., 2033 K STREET N. W., SUITE 800, WASHINGTON, DC, 20006-1021, US

(21) Appl. No.: 10/416,757

(22) PCT Filed: Nov. 5, 2001
(86) PCT No.: PCT/EP01/12790

(30) Foreign Application Priority Data
Nov. 17, 2000 (CH) 2244/00

(51) Int. Cl. G01N 33/53

(52) U.S. Cl. 435/7.1

(57) ABSTRACT

The invention relates to various embodiments of a kit for simultaneous qualitative and/or quantitative determination of numerous analytes, which in particular enables the density of immobilized biological or biochemical or synthetic recognition elements for the determination of said analytes, i.e. the coating density of the measurement area dedicated for these recognition elements, to be referenced. The invention also relates to analytical systems based on the kit according the invention as well as to methods carried out therewith to determine one or more analytes and the use thereof.

The invention relates to various embodiments of a kit for simultaneous qualitative and/or quantitative determination of numerous analytes, which in particular enables the density of immobilized biological or biochemical or synthetic recognition elements for the determination of said analytes, i.e. the coating density of the measurement area dedicated for these recognition elements, to be referenced. The invention also relates to analytical systems based on the kit according to the invention as well as methods carried out therewith to determine one or more analytes and the use thereof.

For the determination of numerous analytes, methods in widespread use at present are in particular those in which different analytes are determined in discrete sample containers or “wells” of so-called microtiter plates. The plates most widely used here are those featuring 8×12 wells on a footprint of typically about 8 cm×12 cm, wherein a volume of some hundred microliters is required for filling a single well. It would be desirable for many applications, however, to determine several analytes simultaneously in a single sample compartment, using a sample volume as small as possible.

In U.S. Pat. No. 5,747,274, measurement arrangements and methods for the early detection of a myocardial infarction by determining several of at least three infarction markers are described, wherein the determination of these markers may be performed in individual sample containers or in a common sample container wherein—as described in the disclosure for the latter case—a single sample container is provided as a continuous flow channel, one demarcation area of which forms a membrane, for example, whereon antibodies for the three different markers are immobilized. However, there is no indication to suggest an arrangement of several such sample containers or flow channels on a common substrate. Furthermore, there is no geometric information with regard to the size of the measurement areas.

In WO 84/01031, U.S. Pat. No. 5,807,755, U.S. Pat. No. 5,837,551, and U.S. Pat. No. 5,432,099, immobilization of specific recognition elements for an analyte in the form of small “spots”, some of which have an area significantly less than 1 mm², on solid substrates is proposed. The purpose of this immobilization geometry is, by binding only a small part of the analyte molecules present, to enable the concentration of an analyte to be determined in a manner which is only dependent on incubation time and (in the absence of a continuous flow) is essentially independent of the absolute sample volume. The measurement arrangements disclosed in the examples are based on fluorescence measurements in conventional microtiter plates. Arrangements are also described here in which spots of up to three different, fluorescently labeled antibodies are measured in a common microtiter plate well. According to the theory set forth in these patent specifications, a minimization of the spot size would be desirable. However, the minimum signal height distinguishable from the background signal would have a limiting effect on the spot size.

Arrays are known which are based on simple glass or microscope plates and have a very high feature density (i.e. density of discrete measurement areas on a substrate, wherein recognition elements for the detection of different analytes are immobilized in these measurement areas). For example, in U.S. Pat. No. 5,445,934 (Affymax Technologies) arrays of oligonucleotides with a density of more than 1000 features per square centimeter are described and claimed. The excitation and set-up of such arrays are based on classical optical arrangements and methods. The whole array may be illuminated at the same time with an expanded excitation light bundle, which leads, however, to relatively low sensitivity, since the proportion of scattered light is relatively large and scattered light or background fluorescence light from the glass substrate is also generated in those areas in which there are no immobilized oligonucleotides for binding of the analyte. To limit excitation and detection to the areas of immobilized features and suppress the generation of light in the adjacent areas, confocal arrangements are used in many cases and the various features sequentially read out by “scanning”. This, however, leads to a longer time period required for read-out of a large array and to a relatively complex optical system. There is no referencing of the measured signals for the detection of different analytes, either with regard to the excitation light intensity available in the measurement areas or with regard to the distribution or (relative) number of immobilized recognition elements. Instead, 2 different samples with different luminescence labels (e.g. with green-emitting and red-emitting labels) are sequentially added to one and the same array, e.g. for expression analysis, in order thereby to compare possible differences in the binding behavior of analytes from different samples on one and the same array.

To achieve lower limits of detection, numerous measurement arrangements have been developed in the last few years, in which detection of the analyte is based on its interaction with the evanescent field, which is associated with light guiding in an optical waveguide, wherein biochemical or biological recognition elements for the specific recognition and binding of the analyte molecules are immobilized on the surface of the waveguide.

When a light wave is coupled into an optical waveguide surrounded by optically rarer media, i.e. media of lower refractive index, the light wave is guided by total reflection at the interfaces of the waveguiding layer. In this arrangement, a fraction of the electromagnetic energy penetrates the media of lower refractive index. This portion is termed the evanescent or decaying field. The strength of the evanescent field depends to a very great extent on the thickness of the waveguiding layer itself and on the ratio of the refractive indices of the waveguiding layer and the surrounding media. In the case of thin waveguides, i.e. layer thicknesses that are the same as or thinner than the wavelength of the light to be guided, discrete modes of the guided light can be distinguished. An advantage of such methods is that the interaction with the analyte is limited to the penetration depth of the evanescent field into the adjacent medium, of the order of magnitude of some hundred nanometers, and interfering signals from the depth of the (bulk) medium can be largely avoided. The first proposed measurement arrangements of this type were based on highly multi-modal, self-supporting single-layer waveguides, such as fibers or plates of transparent plastics or glass, with thicknesses from some hundred micrometers up to several millimeters.

Planar thin-film waveguides have been proposed in order to improve sensitivity and at the same time facilitate mass production. In the simplest case, a planar thin-film waveguide consists of a three-layer system: substrate, waveguiding layer, and superstrate (e.g. the sample to be analyzed), wherein the waveguiding layer has the highest refractive index. Additional intermediate layers can further improve the action of the planar waveguide.

Several methods are known for coupling excitation light into a planar waveguide. The earliest methods used were based on end-face coupling or prism coupling, wherein generally a liquid is introduced between the prism and the waveguide to reduce reflections resulting from air gaps. These two methods are mainly suitable in conjunction with waveguides having relatively large layer thickness—i.e. especially self-supporting waveguides—and a refractive index significantly below 2. By contrast, for the coupling of excitation light into very thin waveguiding layers of high refractive index, the use of coupling gratings is a substantially more elegant method.

In this application, the term “luminescence” describes the spontaneous emission of photons in the range from ultraviolet to infrared, after optical or non-optical excitation, such as electrical or chemical or biochemical or thermal excitation. For example, chemiluminescence, bioluminescence, electroluminescence, and especially fluorescence and phosphorescence are included under the term “luminescence”.

The greater selectivity of signal generation with luminescence-based methods would seem to make these methods better suited to achieving very low detection limits than those based on a change in the effective refractive index (such as grating coupler sensors or methods based on surface plasmon resonance). In this arrangement, luminescence excitation is limited to the penetration depth of the evanescent field into the medium of lower refractive index, i.e. into the immediate vicinity of the waveguiding area, with a penetration depth of the order of some hundred nanometers into the medium. This principle is called evanescent luminescence excitation.

By means of highly refractive thin-film waveguides, in combination with luminescence detection, based on a waveguiding film with a thickness of only a few hundred nanometers on a transparent substrate, the sensitivity has been increased substantially over the last few years. In WO 95/33197, for example, a method is described wherein the excitation light is coupled into the waveguiding film by a relief grating as a diffractive optical element. The surface of the sensor platform is brought into contact with a sample containing the analyte, and the isotropically emitted luminescence from substances which are capable of luminescence and are located within the penetration depth of the evanescent field is measured using suitable measuring devices, such as photodiodes, photomultipliers or CCD cameras. The portion of evanescently excited radiation that has back-coupled into the waveguide can also be coupled out via a diffractive optical element, such as a grating, and measured. This method is described, for example, in WO 95/33198.

A disadvantage of all prior art methods for the detection of evanescently excited luminescence with thin-film waveguides, especially those described in WO 95/33197 and WO 95/33198, is that only one sample at a time can be analyzed on the sensor platform, which is formed as a homogeneous film. In order to perform further measurements on the same sensor platform, elaborate washing or cleaning steps are required each time. This is especially true if an analyte different from the one in the first measurement has to be determined. In the case of an immunoassay, this generally means that the whole immobilized layer on the sensor platform has to be replaced or even that a completely new sensor platform has to be used. In particular, therefore, no simultaneous determinations of multiple analytes can be performed.

For the simultaneous or sequential performance of exclusively luminescence-based, multiple measurements with essentially monomodal, planar inorganic waveguides, arrangements (arrays) have been proposed for example in WO 96/35940, wherein at least two discrete waveguiding areas which are illuminated separately with excitation light are arranged on one sensor platform. However, partitioning of the sensor platform into discrete waveguiding areas has the drawback that the space requirement for discrete measurement areas in discrete waveguiding regions on the common sensor platform is relatively large, and therefore only a relatively low density of different measurement areas (or so-called “features”) can be achieved.

The use of the wording “spatially separated measurement areas” or of “discrete measurement areas”, within the meaning of the present invention, will be defined more precisely in a later section of the invention.

In U.S. Pat. Nos. 5,525,466 and 5,738,992, an optical sensor based on fluorescence excitation in the evanescent field of a self-supporting multimode waveguide, preferably of a fiber-optic type waveguide, is described. In-coupling of excitation light and out-coupling of fluorescence light back-coupled into the multimode waveguide are performed via distal-end in-coupling and out-coupling. Based on the operational principle of such multimode waveguides, the fluorescence signal for analyte determination detected thereby is obtained as a single, integral value for the whole surface interacting with the sample. Mainly for the purpose of signal normalization, for example for taking into account signal-altering surface defects, fluorescent reference compounds are co-immobilized on the sensor surface besides the biochemical or biological recognition elements for the specific recognition and binding of an analyte to be determined. Owing to the underlying sensor principle, however, no locally resolved normalization, but only one acting on the single, integral measurement value is possible. Consequently, the determination of different analytes can also only be performed using labels with different excitation wavelengths or sequentially after the removal of analytes that were previously bound. For these reasons, these arrangements—along with the referencing method described—would appear little if at all suitable for the simultaneous determination of numerous analytes.

In WO 97/35181, methods for the simultaneous determination of one or more analytes are described, wherein patches with different recognition elements are deposited in a “well” formed in a waveguide and brought into contact with a sample solution containing one or more analytes. For calibration purposes, solutions with defined analyte concentrations are applied at the same time to further wells with similar patches. As an example, 3 wells each (for measurement of calibration solutions with high and low analyte concentrations as well as the sample solution) with discrete immobilized recognition elements differing from patch to patch are presented for the simultaneous determination of multiple analytes. There is no evidence to suggest any locally resolved referencing.

In Analytical Chemistry Vol. 71 (1999) 4344-4352, a multianalyte immunoassay on a silicon nitride waveguide is presented. Simultaneous determination of up to three analytes on three channel-like recognition regions (measurement areas) with different biological recognition elements is described. The analytes and tracer antibodies are added as a mixture to a sample cell covering the three measurement areas. The background in each case is determined in advance using a solution without analyte specifically prepared for this purpose. It is not clear from the description whether the background determination is performed on a locally resolved basis or integrally for the different measurement areas. Since the sensor platform is not regenerated, many individual measurements have to be performed, using a new sensor platform each time, to generate a calibration curve. This method, resulting from what is only a small number of measurement areas on a sensor platform and from the assay design, has to be seen as a disadvantage, because the precision of the method is reduced when using different sensor platforms and the duration of the method is considerably increased.

In Analytical Chemistry Vol. 71 (1999) 3846-3852, a multianalyte immunoassay is also presented for the simultaneous determination of three different analytes. Bacillus globigii, MS2 bacteriophages and staphylococcal enterotoxin B are used as examples of analytes from the groups bacteria, viruses, and proteins, wherein antibodies against these analytes have been immobilized in two parallel rows (channels) on a glass plate acting as a (self-supporting multimode) waveguide. In the course of the multianalyte assay subsequently described, a flow cell with flow channels perpendicular to the rows of immobilized recognition elements is placed on the glass plate. The sandwich immunoassays are performed with the sequential addition of washing solution (buffer), of sample containing one or more analytes, of washing solution (buffer), of tracer antibodies (individually or as a cocktail), and of washing solution (buffer). The locally measured fluorescence intensities are corrected by subtraction of the background signal measured adjacent to the measurement areas. Here, too, there is no evidence to suggest local variations in the excitation light intensity to be taken into account. However, this arrangement, too, does not enable the performance of a whole series of measurements for the simultaneous determination of multiple analytes, together with the necessary calibrations, but requires either the use of several different sensor platforms or repetitive, sequential measurements with intermediate regeneration on a platform, which is possible to only a limited extent especially in the case of immunoassays.

In Biotechniques 27 (1999) 778-788, an arrangement of 96 wells, each with 4 arrays of 36 spots (i.e. 144 spots per well in total) on the footprint of a standard microtiter plate (about 8 cm×12 cm) is presented for the development of ELISAs (enzyme-linked immunosorbent assays) based on microarrays. For the purposes of positioning and for checking the efficacy of the reagents used for the enzymatic detection step of the assay by addition of fluorescent “alkaline phosphatase substrate” (ELF®), one row and one column each of the 6×6 measurement areas are reserved for “biotinylated BSA markers”.—Although this arrangement indicates the possibility of a significant increase in the throughput of classical assays (ELISAs); the demonstrated sensitivity (13.4 ng/ml rabbit IgG) would appear unsatisfactory.

In none of the previously discussed documents are suggestions given as to how the immobilization density, i.e. the number of biological or biochemical or synthetic recognition elements applied to a sensor platform per unit area, could be referenced. Both for a reliable manufacture of sensor platforms and also for a precise, quantitative determination of analyte, however, it is very important to know the relative number (in comparisons between different measurement areas) or the absolute number of recognition elements actually present on a sensor platform for a given analyte. In particular, it is to be expected in the case of many different recognition elements on a common sensor platform that these will differ from each other in their adsorption or binding characteristics. Even minor differences in the surface, which is chemically modified for example in batch processes, or during application of the recognition elements for the analyte determination, can lead to marked variations in the immobilization density. Therefore for a commercial manufacture of sensor platforms, for example, the availability of a reliable, nondestructive method of quality assurance, by checking the density of the applied recognition elements, is highly desirable.

Subject of the invention is a kit for the simultaneous qualitative and/or quantitative determination of a multitude of analytes comprising

a sensor platform

at least one array of biological or biochemical or synthetic recognition elements immobilized in discrete measurement areas (d) directly or by means of an adhesion-promoting layer on the sensor platform for specific recognition and/or binding of said analytes and/or for specific interaction with said analytes, wherein for purposes of “referencing the immobilization density”, i.e. for locally resolved determination of the density of immobilized recognition elements in the measurement areas, these recognition elements are associated in each case with a signaling component as label and/or said biological or biochemical or synthetic recognition elements comprise a certain molecular sequence or a certain molecular epitope or a certain molecular recognition group, to which a tracer reagent (referencing reagent), if necessary using a signaling component associated therewith as label, binds for determination of the said density of immobilized recognition elements.

It is advantageous if said certain molecular sequence or said certain molecular epitope or said certain molecular recognition group (such as biotin) is the same for all the different biological or biochemical or synthetic recognition elements immobilized generally in different measurement areas of a segment comprising several measurement areas, with particular preference even for all such elements immobilized in an array of measurement areas. For example an array of measurement areas may comprise discrete measurement areas with numerous different immobilized single-stranded nucleic acids, each having different partial sequences (sub-sequences), for example 10-100 or 10-1000 different partial sequences (sub-sequences), for the recognition and binding of a corresponding number of different nucleic acids complementary to these partial sequences (sub-sequences) as analytes. At the same time, these different immobilized single-strand nucleic acids may possess another partial sequence (sub-sequence) which is common to all of them and which can serve the purpose of “referencing the immobilization density” as described above.

Using the kit according to the invention and the determination method based thereon, it is possible to solve the problem described. It was surprisingly found that, using a kit according to the invention, it is possible to achieve a high level of sensitivity and reproducibility in multianalyte assays for the simultaneous determination of several analytes in a sample similar to the level achieved hitherto in a corresponding number of single assays to determine the individual analytes. At the same time, it was surprisingly found that an optionally used referencing reagent and signaling components that may be associated therewith do not compromise analyte detection.

Within the meaning of the present invention, spatially separated or discrete measurement areas (d) shall be defined by the closed area which is occupied by the biological or biochemical or synthetic recognition elements immobilized thereon, for recognition of an analyte in a liquid sample. Thereby, These areas can have any geometric form, for example the form of points, circles, rectangles, triangles, ellipses or stripes.

In the following, the term “optical transparency” is understood to mean that the material characterized by this property is largely transparent and thus free of absorption at least at one or more excitation wavelengths used for the excitation of one or more luminescences.

A preferred embodiment of the kit according to the invention comprises the immobilized recognition elements in the measurement areas each comprising a general molecular sequence or a general epitope or general molecular recognition group for the purpose of referencing the immobilization density and one or more different sequences or different epitopes or different molecular recognition groups for the recognition and/or binding of different analytes. The said general molecular sequence or said general epitope or said general molecular recognition group for the purpose of “referencing the immobilization density” and a different sequence or different epitope or different molecular recognition group for the recognition and/or binding of different analytes may occur adjacent to one another in a recognition element. To improve accessibility for an analyte to be detected, however, it is preferable if they are sufficiently far away from each other within a recognition element to ensure that the access of an analyte to the sequence specific for its recognition or to the epitope specific for its recognition or specific molecular recognition group of the immobilized recognition element is not hindered. For example, the general and the specific recognition sections (comprising under this name recognition sequence, epitope and molecular recognition group) of an immobilized recognition element may be separated from each other by a so-called molecular spacer (e.g. comprising a chain molecule with hydrocarbon groups). For example, in a kit according to the invention, recognition elements may comprise sections with a general nucleic acid sequence for the purpose of “referencing the immobilization density”, for example in a hybridization step using fluorescently labeled oligonucleotides complementary to this general sequence, and, chemically linked to the general nucleic acid sequence, antibodies or antibody fragments with different recognition epitopes specific in each case for different analytes. Within an immobilized biological or biochemical or synthetic recognition element, several specific recognition sections (according to the definition given hereinbefore) may be present for the recognition and binding of several (different) analytes. These specific recognition sections, as part of the immobilized recognition element, may be arranged consecutively or separated from each other by molecular spacers. In principle, a possible cross-reactivity between the (specific) binding of an analyte to be detected to the specific recognition section intended for it and a possible (nonspecific) binding to the general recognition section of an analyte should be kept as low as possible, ideally at zero. The said general recognition sections (general molecular sequence or general epitope or general molecular recognition group) are preferably to be selected so that the occurrence of a binding partner specific for this general recognition section can be largely excluded in a sample to be added containing the analyte to be detected, provided this binding partner is not added in addition to the sample.

Another possible embodiment of the kit according to the invention comprises, for the said purpose of “referencing the immobilization density”, a referencing reagent for recognition and/or binding to said general sequence or to said general epitope or to said general molecular recognition group of biological or biochemical or synthetic recognition elements immobilized in the same measurement area on the sensor platform being co-immobilized, if necessary in association with said immobilized recognition elements.

For the one preferred embodiment of a kit according to the invention as mentioned hereinbefore, it is further preferred that, for the said purpose of referencing the immobilization density, a referencing reagent for recognition and/or binding to said general sequence or to said general epitope or to the general molecular recognition group of immobilized biological or biochemical or synthetic recognition elements on the sensor platform is applied after immobilization of the biological or biochemical or synthetic recognition elements to the measurement areas of the sensor platform. Said “referencing of the immobilization density”, i.e. the locally resolved determination of the density of immobilized recognition elements in the measurement areas, may be part of a quality control during or after the manufacture of a sensor platform, as part of a kit according to the invention.

Another possibility comprises, for said purpose of referencing the immobilization density, a referencing reagent for recognition and/or binding to said general sequence or to said general epitope or to said general molecular recognition group of the immobilized biological or biochemical or synthetic recognition elements on the sensor platform being applied to the measurement areas of the sensor platform in the course of a detection procedure for the determination of one or more analytes.

Said general molecular sequence or said general epitope or said general molecular recognition group (such as biotin) of the immobilized biological or biochemical or synthetic recognition elements may for example be selected from the group formed by polynucleotides, polynucleotides with synthetic bases, PNAs (“peptide nucleic acids”), PNAs with synthetic bases, proteins, antibodies, peptides, oligosaccharides, lectins, etc.

A preferred embodiment comprises said general sequence of immobilized biological or biochemical or synthetic recognition elements having a length of 5-500, preferably 10-100 bases.

Another preferred embodiment of the kit according to the invention comprises the immobilized recognition elements in the measurement areas in each case being associated with a signal-generating component as label. It can be of further advantage if said signaling component as label changes its signaling properties upon the binding of an analyte to the respective recognition element associated therewith.

A characteristic shared by the various embodiments mentioned of a kit according to the invention is that said different sequences or different epitopes or different molecular recognition groups of immobilized biological or biochemical or synthetic recognition elements are selected from the group comprising nucleic acids (for example DNA, RNA, oligonucleotides) and nucleic acid analogs (e.g. PNA) as well as derivatives thereof with synthetic bases, monoclonal or polyclonal antibodies, peptides, enzymes, aptamers, synthetic peptide structures, glycopeptides, glycoproteins, oligosaccharides, lectins, soluble, membrane-bound proteins and proteins isolated from a membrane, such as receptors, ligands thereof, antigens for antibodies (e.g. biotin for streptavidin), “histidine-tag components” and complex-forming partners thereof, cavities generated by chemical synthesis for hosting molecular imprints, etc. It is also intended that whole cells, cell components, cell membranes or fragments thereof are applied as biological or biochemical or synthetic recognition elements.

It is preferred that a referencing reagent required for certain embodiments of the kit according to the invention comprises a label which is selected from among the group of, for example, luminescence labels, especially luminescent intercalators or “molecular beacons”, absorption labels, mass labels, especially metal colloids or plastic beads, spin labels, such as ESR or NMR labels, and radioactive labels.

It is preferred that said referencing reagent comprises a luminescence label or absorption label. In particular, said referencing reagent may also comprise an intercalator or a “molecular beacon”. It is preferred in this case that said intercalator or “molecular beacon” changes its signaling properties in the presence of the referencing reagent.

Before or during an analytical detection procedure, said referencing reagent may be cleaved off or remain associated with the recognition elements.

A further advantageous embodiment of the kit according to the invention comprises said referencing reagent including a component from among the group formed by, for example, polynucleotides, polynucleotides with synthetic bases, PNAs (“peptide nucleic acids”), PNAs with synthetic bases, proteins, antibodies, biotin, streptavidin, peptides, oligosaccharides, lectins, etc.

A further characteristic shared by the mentioned embodiments of the kit according to the invention is that the quantitative and/or qualitative detection of the said multitude of analytes comprises the use of one or more signaling components as labels, which may be selected from among the group that is formed by, for example, luminescence labels, especially luminescent intercalators or “molecular beacons”, absorption labels, mass labels, especially metal colloids or plastic beads, spin labels, such as ESR or NMR labels, and radioactive labels.

It is preferred that the label of the referencing reagent and/or an analyte detection optionally based on absorption and/or luminescence detection is based on the use of labels with the same or different absorption and/or luminescence wavelengths.

A special embodiment, based on the recognition elements immobilized in the measurement areas, in each case with an associated signaling component as label, comprises the said label also serving for analyte detection in addition to referencing the immobilization density of the recognition elements. For example, the said label may be a fluorescent intercalator which, bound to a single-stranded nucleic acid as immobilized recognition element, emits a very weak, but nevertheless measurable signal, from which the density of the recognition elements immobilized in the corresponding measurement areas can be determined. On hybridization with a (single-stranded) nucleic acid in an added sample as analyte, which is at least partly complementary, especially in the region of the immobilized intercalator, a marked increase may occur in the fluorescence intensity of this intercalator, on the basis of which the analyte concerned is then qualitatively and/or quantitatively detected in this measurement area.

The detection of analytes is preferably based on determining the change in one or more luminescences.

A possible embodiment comprises the excitation light from one or more light sources for generating the signals of signaling components for the purpose of chemical referencing and/or for the detection of one or more analytes being delivered in an epi-illumination array.

For numerous embodiments, it is preferred that the sensor platform material which is in contact with the measurement areas is transparent or absorbent for at least one excitation wavelength within a depth of at least 200 nm from the measurement area.

Other embodiments comprise the excitation light from one or more light sources for generating the signals of signaling components for the purpose of referencing the immobilization density and/or for the detection of one or more analytes being delivered in a transillumination configuration.

In many cases, it is of advantage if the sensor platform material is transparent for at least one excitation wavelength.

A preferred embodiment of a kit according to the invention comprises the sensor platform being provided as an optical waveguide which is preferably essentially planar. The sensor platform here preferably comprises a material from the group formed by silicates, e.g. glass or quartz, transparent thermoplastic or moldable plastic, for example polycarbonate, polyimide, acrylates, especially polymethylmethacrylate, or polystyrenes.

Characteristic for an especially preferred embodiment of a kit according to the invention is, that the sensor platform comprises an optical thin-film waveguide with a layer which is transparent for at least one excitation wavelength (a) on a layer which is likewise transparent for at least this excitation wavelength (b) with a lower refractive index than layer (a).

Various embodiments of such sensor platforms and methods for the detection of one or more analytes using such sensor platforms are described in detail for example in patents U.S. Pat. No. 5,822,472, U.S. Pat. No. 5,959,292 and U.S. Pat. No. 6,078,705 as well as in patent applications WO 96/35940, WO 97/37211, WO 98/08077, WO 99/58963, PCT/EP 00/04869 and PCT/EP 00/07529. Embodiments of a kit according to the invention with the embodiments of sensor platforms described in these patents or patent applications, as an integral part of a kit according to the invention, and methods to detect one or more analytes using a kit according to the invention with such sensor platforms are likewise the subject of the present invention.

For a kit according to the invention, with an optical waveguide as sensor platform, it is preferred that the excitation light from one or more light sources is coupled into the optical waveguide using a method selected from the group formed by end-face (distal end) coupling, coupling via attached optic fibers as lightguides, prism coupling, grating coupling or evanescent coupling by overlapping of the evanescent field of said optical waveguide with the evanescent field of a further waveguide brought into near-field contact therewith.

In general, the aim is to avoid as far as possible generating reflections of delivered excitation light, since these usually lead, in an essentially disadvantageous manner, to an increase in background signals. For example, the occurrence of reflections can be expected in principle, when the excitation light passes through optical boundary surfaces of media with different refractive indices. It is therefore of advantage if the in-coupling of the excitation light from one or more light sources into the optical waveguide is performed by means of an optical coupling element which is in contact therewith and which is selected from the group of optical fibers as lightguidess, prisms, if necessary using a refractive index-matching liquid, and grating couplers.

Especially preferred is an embodiment of the kit according to the inventions which comprises the excitation light from one or more light sources being in-coupled into layer (a) by means of one or more grating structures (c) modulated in layer (a).

Suitable geometric arrangements of such grating structures for a sensor platform as part of a kit according to the invention are in turn described for example in patents U.S. Pat. No. 5,822,472, U.S. Pat. No. 5,959,292 and U.S. Pat. No. 6,078,705 as well as in patent applications WO 96/35940, WO 97/37211, WO 98/08077, WO 99/58963, PCT/EP 00/04869 and PCT/EP 00/07529 and, as integral part of a kit according to the invention, are likewise an object of the present invention.

For numerous embodiments, it is preferred that the sensor platform comprises uniform, non-modulated areas of layer (a), which are preferably arranged in the direction of propagation of the excitation light in-coupled into layer (a) via a grating structure (c) and guided in layer (a).

In general, grating structures (c) can be used for the in-coupling of excitation light towards measurement areas (d) and/or for the out-coupling of luminescence light back-coupled into layer (a). As a general embodiment, therefore, the sensor platform comprises numerous grating structures (c) of similar or different periods, with optionally adjacent uniform, non-modulated regions of layer (a) on a common, continuous substrate.

For the assay applications using a kit according to the invention, it is generally advantageous to in-couple a suitable excitation light by means of a grating structure (c), adjacent to which, in the direction of propagation of the in-coupled light guided in layer (a), is located a nonmodulated region of layer (a) bearing numerous measurement areas in an array, on which the detection of different analytes is performed. It is advantageous if another grating structure with a further array of measurement areas adjacent to it is located behind (in the direction of propagation of the guided light) this first described region, etc. After passing through a nonmodulated region, the light guided in layer (a) will in each case be out-coupled again. In the direction perpendicular to the direction of propagation of the guided light (i.e. parallel to the grating lines) further arrays of measurement areas will be provided. It is therefore preferred that a dedicated grating structure (c) for out-coupling of the guided excitation light is provided following, in direction of propagation of the in-coupled excitation light, subsequent to each array of measurement areas, wherein, perpendicular to the direction of propagation of the in-coupled excitation light, individual grating structures for different arrays can be provided, or these grating structures can also extend in this direction (perpendicular to the direction of propagation of the in-coupled excitation light) over the whole sensor platform. that the in-coupling grating for an array following in direction of propagation of the excitation light guided in layer (a) of a sensor platform is used as an out-coupling grating for the excitation light that has been in-coupled at the in-coupling grating of the aforementioned array preceding in said direction of propagation.

For certain applications, for example when using two or more luminescence labels with different excitation wavelengths, it is advantageous if the sensor platform comprises a superposition of two or more grating structures of different periodicities for the in-coupling of excitation light of different wavelengths, the grating lines being parallel or not parallel, preferably not parallel, to each other, wherein in the case of two superimposed grating structures their grating lines are preferably perpendicular to each other.

The partitioning of the sensor platform into sections with grating structures modulated therein and adjacent nonmodulated sections means in practice that the area requirements for a single array of measurement areas between two consecutive grating structures (including at least one grating structure dedicated for said array) cannot be reduced below a certain minimum which, with the current technical options available for the manufacture of grating structures and for the in-coupling of a suitable excitation light bundle, is of the order of 0.1 mm² to 1 mm². It is therefore advantageous, especially for arrangements in which a large number of small-area arrays is desired, if a grating structure (c) or a superposition of several grating structures in layer (a) is essentially modulated across the whole area of the sensor platform.

In a further embodiment of the invention it is preferred that the sensor platform is furnished with optically or mechanically recognizable markings to facilitate adjustment in an optical system and/or for connection to sample compartments as part of an analytical system.

If an autofluorescence of layer (b) cannot be excluded, especially if it comprises a plastic such as polycarbonate, or also in order to reduce the effect of the surface roughness of layer (b) on the light transmission in layer (a), it may be advantageous if an intermediate layer is deposited between layers (a) and (b). For this reason, a further embodiment of the arrangement according to the invention comprises the application of an additional optically transparent layer (b′) with a lower refractive index than that of layer (a) and with a thickness of 5 nm-10000 nm, preferably 10 nm-1000 nm, between the optically transparent layers (a) and (b) and in contact with layer (a).

The simplest method of immobilization of the biological or biochemical or synthetic recognition elements consists in physical adsorption, for example as a result of hydrophobic interaction between the recognition elements and the baseplate. However, the extent of these interactions may be substantially altered by the composition of the medium and its physicochemical properties, such as polarity and ionic strength. Especially when different reagents are sequentially added in a multistep assay, the adhesion of the recognition elements after only adsorptive immobilization is often insufficient. In a preferred embodiment of the kit according to the invention, the adhesion is improved by deposition of an adhesion-promoting layer (f) on the sensor platform for the immobilization of biological or biochemical or synthetic recognition elements. Especially when biological or biochemical recognition elements are to be immobilized, the adhesion-promoting layer can also serve to improve the “biocompatibility” of their environment, i.e. to preserve the binding capacity of the recognition elements, in comparison with the binding capacity in their natural biological or biochemical environment, and to avoid denaturation. It is preferred if the adhesion-promoting layer (f) has a thickness of less than 200 nm, preferably of less than 20 nm. Many materials can be used to produce the adhesion-promoting layer. Without any restriction, it is preferred if the adhesion-promoting layer (f) comprises one or more chemical compounds from the groups comprising silanes, epoxides, functionalized, charged or polar polymers, and “self-organized passive or functionalized monolayers or multiple layers”.

A further essential aspect of the kit according to the invention is that the biological or biochemical or synthetic recognition elements are immobilized in discrete measurement areas (d). These discrete measurement areas (d) may be generated by laterally selective deposition of biological or biochemical or synthetic recognition elements on the sensor platform. Numerous known methods can be used for the deposition. Without loss of generality, it is preferred if the biological or biochemical or synthetic recognition elements are deposited on the sensor platform by one or more methods from the group of methods formed by “ink jet spotting”, mechanical spotting by means of pin, pen or capillary, “micro contact printing”, fluidic contact of the measurement areas with the biological or biochemical or synthetic recognition elements through their application in parallel or intersecting microchannels, upon exposure to pressure differences or to electric or electromagnetic potentials, and photochemical or photolithographic immobilization methods.

A further special embodiment of the kit according to the invention comprises the density of the recognition elements immobilized in discrete measurement areas for the detection of different analytes on different measurement areas being selected in such a way that the luminescence signals on determination of different analytes in a common array are of similar order of magnitude, i.e. that, if necessary, the related calibration curves for the analyte determinations to be performed at the same time may be recorded without a change in the settings of the electronic or opto-electronic system.

Another advantageous variant of the kit according to the invention comprises arrays of measurement areas being divided into segments of one or more measurement areas for the determination of analytes and regions between these measurement areas or additional measurement areas for the purpose of the physical referencing, for example, of the excitation light intensity available in the measurement areas or of the influence of changes in external parameters, such as temperature, and for the purpose of referencing the influence of additional physicochemical parameters, such as nonspecific binding of components of an applied sample to the sensor platform.

For certain applications, in which the main focus concerns, for example, questions of the reproducibility of results using a multitude of arrays on a common sensor platform, it is advantageous if two or more arrays have a similar geometric arrangement of measurement areas and/or segments of measurement areas for determining similar analytes on these arrays.

It can likewise be of advantage, especially for investigating the reproducibility of measurements on different measurement areas, if one or more arrays comprise segments of two or more measurement areas with similar biological or biochemical or synthetic recognition elements within the segment for analyte determination or referencing.

In other applications, it is essential to minimize the influences of systematic errors on the results, as may arise for example from a replication of similar structures on a common sensor platform. It may be of advantage in this case, for example, if two or more arrays have different geometric arrangements of measurement areas and/or segments of measurement areas for the determination of similar analytes on these arrays.

The kit according to the invention with a multitude of measurement areas in discrete arrays, of which many may in turn be arranged on a common sensor platform, offers the possibility of conducting many kinds of duplication or multiple performance of similar measurements using relatively small quantities of sample solutions, reagents or optionally calibration solutions on one and the same platform under largely identical conditions. Thus, for example, statistical data can be generated in a single measurement which by conventional means would require a large number of individual measurements with a correspondingly longer total measurement time and consumption of greater amounts of samples and reagents. It is preferred if two or more identical measurement areas within a segment or an array are provided in each case for the determination of each analyte or for referencing. Said identical measurement areas can be arranged here, for example, in a continuous row or column or diagonal of an array or a segment of measurement areas. The aspects of referencing may be related to physical or physicochemical parameters of the sensor platform, such as local variations of the excitation light intensity (see also below), as well as effects of the sample, such as its pH, ionic strength, refractive index, temperature, etc.

For other applications, however, it may also be advantageous if said identical measurement areas are distributed statistically within an array or a segment of measurement areas.

In general, the immobilized recognition elements are selected in such a way that they recognize and bind the analyte to be determined with a specificity as high as possible. In general, however, it must be expected that also a nonspecific adsorption of analyte molecules occurs on the surface of the baseplate, especially if there are still empty reactive sites between the recognition elements immobilized in the measurement areas It is therefore preferred if regions between the laterally separated measurement areas (d) are “passivated” in order to minimize nonspecific binding of analytes or their tracer compounds, i.e. if compounds are deposited between the laterally separated measurement areas (d) which are “chemically neutral” to the analyte, preferably for example compounds from groups comprising albumins, especially bovine serum albumin or human serum albumin, casein, nonspecific polyclonal or monoclonal, heterologous or empirically nonspecific antibodies for the analyte or analytes to be determined (especially for immunoassays), detergents (such as Tween 20®), fragmented natural or synthetic DNA not hybridizing with polynucleotides to be analyzed, such as extract from herring or salmon sperm (especially for polynucleotide hybridization assays), or also uncharged but hydrophilic polymers, such as polyethylene glycols or dextrans.

By the addition of reducing reagents, such as sodium borohydrate, it is also possible to passivate a surface (comprising for example poly-L-lysine or functionalized silanes, for example with aldehyde or epoxy groups) that has been activated (for immobilization of the biological or biochemical or synthetic recognition elements).

As described hereinbefore, for many if not most applications such an embodiment of the kit according to the invention is of advantage in which an adhesion-promoting layer is applied before immobilization of the biological or biochemical or synthetic recognition elements on the sensor platform. Such embodiments are preferred here which comprise the passivation of regions between discrete measurement areas in order to minimize the nonspecific binding of analytes or tracer substances thereof being achieved by the application of said adhesion-promoting layer to the sensor platform without the application of additional substances.

The kit according to the invention may comprise a very large number of measurement areas. It is preferred if up to 100,000 measurement areas are provided in a 2-dimensional arrangement and a single measurement area occupies an area of 0.001 mm²-6 mm². The number of measurement areas on a sensor platform as part of the kit according to the invention is preferably more than 100, more preferably more than 1000, and even more preferably more than 10,000.

A further subject of the invention is an embodiment of the kit according to the invention wherein the upper surface of the sensor platform, with the measurement areas generated thereon, is combined with a further body over the optically transparent layer (a) in such a way that one or more cavities are formed between the sensor platform as baseplate and said body for the generation of one or more sample compartments which are fluidically sealed against one another and each of which comprises one or more measurement areas or segments or arrays of measurement areas.

A preferred embodiment in this case comprises the sample compartments as flow cells fluidically sealed against one another being formed in each case with at least one inlet and at least one outlet and optionally at least one outlet of each flow cell in addition leading to a reservoir fluidically connected to this flow cell to receive fluid exiting the flow cell.

It is advantageous in this case if the optional additional reservoir for receiving liquid exiting the flow cell is provided as a recess in the outer wall of the body connected with the sensor platform as baseplate.

There are various technical options for creating recesses between the sensor platform as baseplate and the connected body. In one possible arrangement, three-dimensional structures with the pitch of the flow cell arrays to be generated are formed on the sensor platform as baseplate. These structures on the baseplate may, for example, form the walls or parts thereof, such as bases, between the adjacently arranged flow cells, which are created by the combination of the baseplate with a correspondingly formed body. To generate the array of flow cells, it is also possible to provide recesses in the sensor platform for creating the cavities between the sensor platform as baseplate and the body combined therewith.

A further embodiment comprises the formation of recesses in said body for the creation of cavities between the baseplate and the connected body. For this embodiment, it is preferred if the baseplate is essentially planar.

The body to be combined with the baseplate in order to create the array of flow cells may consist of a single workpiece. Another embodiment comprises the body connected to the baseplate being formed from several parts, wherein the combined parts of said body preferably form an irreversibly combined unit.

It is preferred if the body combined with the baseplate comprises auxiliary arrangements facilitating the combination of said body and the baseplate.

The arrangement preferably comprises a large number, i.e. 2-2000, preferably 2-400, especially preferably 2-100 sample compartments.

For example, for applications in which the samples and/or additional reagents are to be supplied directly using a dispenser, it is preferred if the sample compartments are open on that side of the body combined with the sensor platform as baseplate which lies opposite the measurement areas.

It is preferred if the pitch (geometrical arrangement in rows and/or columns) of the sample compartments matches the pitch of the wells on a standard microtiter plate.

A further embodiment of the arrangement of sample compartments as part of the kit according to the invention comprises its closure with an additional covering top, for example a film, a membrane or a cover plate.

By varying the size of the base areas and the depth of the recesses, the capacity of the flow cells can be varied within a wide range so that the inner volume of each sample compartment is typically 0.1 μl-1000 μl, preferably 1 μl-20 μl. Thereby, the inner volumes of different flow cells of an arrangement here may be similar or different.

It is preferred if the depth of the cavities between the sensor platform as baseplate and the body combined with said baseplate is 1-1000 μm, preferably 20-200 μm. The size of the cavities of an array may be uniform or different and the base areas may be of any shape, preferably rectangular or polygonal or of any other geometry. The lateral dimensions of the base areas may also vary within a wide range, wherein the base areas of the cavities between the baseplate and the body combined with said baseplate are typically 0.1 mm²-200 mm², preferably 1 mm²-100 mm². The corners of the base areas are preferably rounded. Rounded corners have a favorable effect on the flow profile and facilitate the removal of any gas bubbles that might be formed or prevent their formation.

For simultaneous dosing of samples or reagents into a multitude of sample compartments, multichannel pipettors for manual or automatic reagent application can be used, wherein the individual pipettes are arranged in one-dimensional or two-dimensional arrays, provided the arrangement of sample compartments as part of the kit according to the invention is provided with inlets arranged in the corresponding pitch. It is therefore preferred if the pitch (geometrical arrangement in rows and columns) of the arrangement matches the pitch of the wells on a standard microtiter plate. Thereby, an arrangement of 8×12 wells with a (center-to-center) distance of about 9 mm is established as the industrial standard. Smaller arrays with, for example, 3, 6, 12, 24 and 48 wells, arranged at the same distance, are compatible with this standard. Several arrangements of sample compartments according to the invention with such small arrays of flow cells may also be combined in such a way that the individual inlets of said flow cells are arranged at an integral multiple of the distance of about 9 mm.

For some time also plates with 384 and 1536 wells, as integral multiples of 96 wells on the same foot print at a correspondingly reduced well-to-well distance, are used, which shall also be called standard microtiter plates. By adapting the pitch of the sample compartments in the arrangement according to the invention, including the inlets and outlets of each flow cell, to these standards, numerous commercially established and available laboratory pipettors and robots may be used for sample dosing.

The outer base dimensions of the arrangement of sample compartments, as part of the kit according to the invention, preferably correspond to the footprint of these standard microtiter plates.

A further special embodiment of the invention is an arrangement of, for example, 2 to 8 sample compartments, as part of the kit according to the invention, with the aforementioned properties, in a column or, for example, 2 to 12 sample compartments in a row which are combined in turn with a carrier (“meta-carrier”) with the dimensions of standard microtiter plates in such a way that the pitch (geometrical arrangement in rows or columns) of the inlets of the sample compartments matches the pitch of the wells on a standard microtiter plate.

Several such columns or rows of sample compartments may be combined with a single such meta-carrier in such a way that the pitch (geometric arrangement in rows or columns) of the flow cell inlets matches the pitch of the wells on a standard microtiter plate, i.e. an integral multiple of 9 mm (corresponding to 96-well plate) or of 4.5 mm (corresponding to 384-well plate, see above) or of 2.25 mm (corresponding to 1536-well plate, see above).

However, the sample compartments may of course also be arranged in a different pitch.

The materials for the body combined with the sensor platform as baseplate and an optionally used additional cover plate must satisfy the requirements for the planned use of the arrangement in each case. Depending on the specific application, these requirements relate to chemical and physical resistance, for example, to acidic or basic media, salts, alcohols or detergents as components of aqueous solutions, or formamide, thermal resistance (e.g. between −30° C. and 100° C.), the most as similar possible thermal expansion coefficients of baseplate and the body combined therewith, optical properties (such as nonfluorescence and reflectivity), mechanical workability, etc. The material of the body combined with the baseplate and of an optional additional cover plate is preferably selected from the same group as the material of the “meta-carrier”. The aforementioned components in this case (the body combined with the sensor platform as baseplate and the cover plate) may be composed of a uniform material or may comprise a mixture of different materials or a composition thereof fitted together in layers or laterally, wherein the materials may substitute each other.

A very important aspect of the present invention concerns the possibilities for locally resolved referencing of the available excitation light intensity. In conventional arrangements, with excitation light delivered in an epi-illumination or transillumination configuration, the available excitation light intensities of an irradiated area are mainly determined by the excitation light density in the cross-section of the excitation light bundle. In this case, local variations of the properties of the illuminated surface (such as a glass plate) have only a secondary influence. However, in the arrangement of the kit according to the invention, local variations in the physical parameters of the sensor platform, such as the in-coupling efficiency of the grating structure (c) for the in-coupling of the excitation light into the optically transparent layer (a), or local variations in the propagation losses of a guided mode in the optically transparent layer (a) are of crucial importance. Such embodiments of a kit according to the invention, wherein the means for locally resolved referencing of the excitation light intensity available in the measurement areas comprise the simultaneous or sequential generation of an image of the light emanating from the sensor platform at the excitation wavelength, thus form a further important subject of the invention. A precondition here is that the losses by scattered light are essentially proportional to the locally guided light intensity. The losses by scattered light are mainly determined by the surface roughness and homogeneity of the optically transparent layer (a) and of the substrate located beneath (optically transparent layer (b)). In particular, this type of referencing allows a local decrease in the locally available excitation light intensity in the direction of its propagation to be taken into account, if this decrease occurs, for example, as a result of an absorption of excitation light caused by a high local concentration of molecules in the evanescent field of the layer (a), which are absorbent at the excitation wavelength.

However, the assumption of the proportionality of the emitted scattered light to the intensity of the guided light is not valid at those locations where an emission/out-coupling occurs as a result of local macroscopic scattering centers in contact with the layer (a). At these locations, the intensity of emitted scattered light is much greater than proportional to the intensity of guided light. It is therefore also advantageous if the arrangements for locally resolved referencing of the excitation light intensity available in the measurement areas comprise the simultaneous or sequential generation of an image of the light emanating from the sensor platform at the luminescence wavelength. The two methods of course can also be combined. In the generation of a reference image, various influences of the imaging optics on the recording of measurement signals should be excluded. For this reason, an image of the excitation light emanating from the sensor platform is preferably generated via the same optical path as that used to record the luminescences emanating from the measurement areas.

Another embodiment comprises the arrangements for locally resolved referencing of the excitation light intensity available in the measurement areas being the simultaneous or sequential generation of an image of the light emanating from the sensor platform at an excitation wavelength other than that used for excitation of a luminescence.

It is further preferred if the local resolution of the image for referencing of the excitation light emanating from the sensor platform is below 100 μm on the sensor platform, and preferably below 20 μm. On the assumption of such a local resolution, it is further preferred if the arrangements for locally resolved referencing of the excitation light intensity available in the measurement areas comprise the determination of the background signal at the relevant luminescence wavelength between or adjacent to the measurement areas.

A preferred embodiment of the kit according to the invention comprises the locally resolved referencing of the excitation light intensity available in the measurement areas being performed by means of “luminescence marker spots”, i.e. determination of luminescence intensity from measurement areas with pre-immobilized luminescently labeled molecules (i.e. molecules have already been deposited in these measurement areas before dosing of a sample). In this case, the “luminescence marker spots” are preferably applied in a pattern covering the whole sensor platform.

As described in more detail hereinbelow, preferably locally resolving detectors, such as CCD cameras (CCD: charge-coupled device) are used for signal detection. Characteristic for these detectors is, that their photo-sensitive elements (pixels) deliver a certain (essentially thermally caused) background signal determining the lower threshold for the detection of a local light signal, and that they also have a maximum capacity (saturation) for the detection of high light intensities. The difference between these threshold values, at a given exposure time, determines the dynamic range for signal detection. Both the luminescence signals to be determined for analyte detection and the reference signals should lie within this dynamic range. It is advantageous here if both signals are of a similar order of magnitude, i.e. for example, if they do not differ by more than one or two orders of magnitude. According to the invention, this may be achieved, for example, by selecting the density of the luminescently labeled molecules within a “luminescence marker spot”, upon mixing with similar, but unlabeled molecules during immobilization, in such a way that the luminescence intensity from the regions of the “luminescence marker spots” is of similar order of magnitude as the luminescence intensity from the measurement areas intended for analyte determination.

The density and concentration of the luminescently labeled molecules within the “luminescence marker spots” in an array should preferably be uniform over the entire sensor platform.

In this type of referencing, the local resolution is essentially determined by the density of the “luminescence marker spots” within an array and/or over the entire sensor platform. The distance between and/or the size of different “luminescence marker spots” are preferably matched to the desired local resolution in the determination of luminescence intensities from the discrete measurement areas.

Each array on the sensor platform preferably comprises at least one “luminescence marker spot”. It is advantageous if there is at least one adjacent “luminescence marker spot” for each segment of measurement areas for the determination of an analyte. It is especially advantageous if each measurement area is surrounded by “luminescence marker spots”.

There are numerous possibilities for the geometric arrangement of the “luminescence marker spots” within an array or on the sensor platform. One possible arrangement, for example, consists in that each array comprises a continuous row and/or column of “luminescence marker spots” in parallel and/or perpendicular to the direction of propagation of the in-coupled excitation light, for determination of the two-dimensional distribution of the in-coupled excitation light in the region of said array.

It is intended that the arrangements for locally resolved referencing of the excitation light intensity available in the measurement areas comprise the determination of an average of multiple locally resolved reference signals. In the case of nonstatistically, but systematically varying excitation light intensities in the form of a gradient over certain distances, interpolation on the expected value of excitation light intensity of a measurement area lying between different areas for locally resolved referencing may be advantageous.

A further essential feature of the kit according to the invention comprises arrangements to calibrate recorded luminescence signals in the presence of one or more analytes. A possible embodiment comprises said arrangements for the calibration of luminescences generated as a result of the binding of one or more analytes or as a result of specific interaction with one or more analytes being the addition of calibration solutions with known concentrations of the analytes to be determined to a predetermined number of arrays. It is possible, for example, that 8-12 arrays of a sensor platform are intended for calibration purposes.

With the large number of measurement areas on a sensor platform, the kit according to the invention permits a further possibility for calibration which has not been hitherto described. This possibility consists in the fact that it is essentially not necessary to add a large number of calibration solutions with different, known concentrations to one or more arrays, but to immobilize the biological or biochemical or synthetic recognition elements used for analyte determination in known, but different local concentration in the measurement areas intended for calibration purposes. Just as a calibration curve can be generated by applying various calibration solutions of different analyte concentrations on an array with recognition elements at a single uniform immobilization density so too is it possible in principle to generate such a standard curve reflecting the binding activity and frequency of the binding events between an analyte and its tracer elements by applying a single calibration solution to an array with recognition elements at a different immobilization density. It is essential for the feasibility of this simplified type of calibration that the precise binding behavior between an analyte and its recognition elements is known and that the variation, i.e. the difference between the lowest and the highest immobilization density in the measurement areas dedicated for an analyte is sufficient for the calibration to cover the entire application range of an assay intended for analyte detection.

A further subject of the invention is therefore a kit which comprises several measurement areas with biological or biochemical or synthetic recognition elements immobilized therein at a different, controlled density being provided in each case in one or more arrays for the determination of an analyte that is common to these measurement areas. It is especially preferred here if a calibration curve for this analyte can be established already with the application of a single calibration solution to an array comprising biological or biochemical or synthetic recognition elements for said analyte immobilized in different measurement areas of that array at a sufficiently large “variation” of different controlled density and with known concentration dependence of the signals indicative for the binding between said analyte and said biological or biochemical or synthetic recognition elements.

A further subject of the invention is the use of a kit according to one of the said embodiments in an analytical system for the determination of one or more luminescences.

A further subject of the invention is an analytical system with any embodiment of the kit according to the invention comprising additionally at least one detector for the recording of one or more luminescences.

A further subject of the invention is an analytical system for determining one or more luminescences with

at least one excitation light source

a kit according to the invention and

at least one detector for recording the light emanating from one or more measurement areas (d) on the sensor platform.

A possible embodiment of the analytical system comprises the excitation light being delivered to the measurement areas in an epi-illumination or transillumination arrangement.

A preferred embodiment of the analytical system according to the invention comprises the excitation light which emanates from at least one excitation light source being essentially parallel and being delivered at the resonance angle for in-coupling into the optically transparent layer (a) onto a grating structure (c) modulated in layer (a).

One possibility comprises the excitation light from at least one light source being expanded to an essentially parallel bundle of light rays by means of an expansion lens and delivered at the resonance angle for in-coupling into the optically transparent layer (a) onto a grating structure (c) with a large surface area modulated in layer (a).

The luminescence light is preferably detected in such a way that the out-coupled luminescence light from a grating structure (c) or (c′) is recorded by the detector as well.

Numerous other suitable analytical systems with a kit according to the invention as a part thereof are described for example in patents U.S. Pat. No. 5,822,472, U.S. Pat. No. 5,959,292 and U.S. Pat. No. 6,078,705 as well as in patent applications WO 96/35940, WO 97/37211, WO 98/08077, WO 99/58963, PCT/EP 00/04869 and PCT/EP 00/07529 and in the use of a kit according to the invention are likewise a subject of the present invention.

It is further preferred if the analytical system according to the invention comprises in addition supply means for bringing the one or more samples into contact with the measurement areas on the sensor platform.

A further embodiment of the analytical system comprises compartments being provided for reagents which are wetted during the procedure for the detection of one or more analytes and brought into contact with the measurement areas.

A further subject of the invention is a method for the simultaneous qualitative and/or quantitative detection of a multitude of analytes using a kit according to the invention as described in one of the said embodiments and/or using an analytical system according to the invention as described in one of the said embodiments, wherein, for the purpose of “referencing the immobilization density”, i.e. for locally resolved determination of the density of immobilized biological or biochemical or synthetic recognition elements in the measurement areas, these recognition elements are associated with a signaling component as label and/or have a certain molecular sequence or a certain molecular epitope or a certain molecular recognition group to which a detection reagent (referencing reagent) binds, optionally with an associated signaling component as label, for the determination of said density of immobilized recognition elements, and the signals of said signaling components are recorded in a locally resolved manner. Determination of the immobilization density of biological or biochemical or synthetic recognition elements on the sensor platform and the detection of said multitude of analytes can be performed here independently of each other. In particular, the determination of the immobilization density of biological or biochemical or synthetic recognition elements on the sensor platform may form part of the quality control during or after the manufacture of said sensor platform.

Charcetristic for a preferred embodiment of the kit according to the invention is that the immobilized recognition elements in the measurement areas each comprise a general molecular sequence or a general epitope or general molecular recognition group for the purpose of referencing the immobilization density and a different sequence or different epitope or different molecular recognition group for the recognition and/or binding of different analytes. The said general molecular sequence or said general epitope or said general molecular recognition group for the purpose of referencing the immobilization density and a different sequence or different epitope or different molecular recognition group for the recognition and/or binding of different analytes may occur adjacent to one another in a recognition element. To improve accessibility for an analyte to be detected, however, it is preferable if they are sufficiently far away from each other within a recognition element to ensure that the access of an analyte to the sequence specific for its recognition or to the epitope specific for its recognition or specific molecular recognition group of the immobilized recognition element is not hindered. For example, the general and the specific recognition sections (in this wording 0comprising recognition sequence and epitope) of an immobilized recognition element may be separated from each other by a so-called molecular spacer (e.g. comprising a chain molecule with hydrocarbon groups). For example, in a method according to the invention using a kit according to the invention, recognition elements may comprise sections with a general nucleic acid sequence for the purpose of “referencing the immobilization density”, for example in a hybridization step using fluorescence-labeled oligonucleotides complementary to this general sequence, and antibodies or antibody fragments chemically linked to the general nucleic acid sequence with different recognition epitopes specific in each case for different analytes. In principle, a possible cross-reactivity between the (specific) binding of an analyte for detection to the specific recognition section intended for it and a possible (nonspecific) binding to the general recognition section of an analyte should be kept as low as possible, ideally at zero. The said general recognition sections (general molecular sequence or general epitope or general molecular recognition group) are preferably to be selected so that the occurrence of a binding partner specific for this general recognition section can be largely excluded in a sample to be added containing the analyte to be detected, provided this binding partner is not added in addition to the sample.

Another possible embodiment of the kit according to the invention comprises, for the said purpose of referencing the immobilization density, a referencing reagent for recognition and/or binding to said general sequence or to said general epitope or to said general molecular recognition group of biological or biochemical or synthetic recognition elements immobilized in the same measurement area on the sensor platform being co-immobilized, if necessary in association with said immobilized recognition elements.

For the preferred embodiment of a method according to the invention as mentioned hereinbefore, it is further preferred that, for the said purpose of referencing the immobilization density, a referencing reagent for recognition and/or binding to said general sequence or to said general epitope or to the general molecular recognition group of immobilized biological or biochemical or synthetic recognition elements on the sensor platform is applied after immobilization of the biological or biochemical or synthetic recognition elements to the measurement areas of the sensor platform. Said “referencing of immobilization density”, i.e. locally resolved determination of the density of immobilized recognition elements in the measurement areas, may be part of a quality control during or after the manufacture of a sensor platform, as part of a method according to the invention.

Another possibility comprises, for said purpose of referencing the immobilization density, a referencing reagent for recognition and/or binding to said general sequence or to said general epitope of the immobilized biological or biochemical or synthetic recognition elements on the sensor platform being applied to the measurement areas of the sensor platform in the course of a detection procedure for the determination of one or more analytes.

Said general molecular sequence or said general epitope or said general molecular recognition group (such as biotin) of the immobilized biological or biochemical or synthetic recognition elements may for example be selected from the group comprising polynucleotides, polynucleotides with synthetic bases, PNAs (“peptide nucleic acids”), PNAs with synthetic bases, protein, antibodies, peptides, oligosaccharides, lectins, etc.

A preferred embodiment comprises said general sequence of immobilized biological or biochemical or synthetic recognition elements having a length of 5-500, preferably 10-100 bases.

Another preferred embodiment of the method according to the invention comprises the immobilized recognition elements in the measurement areas in each case being associated with a signal-generating component as label. It can be of further advantage if said signaling component as label changes its signaling properties in the binding of an analyte to the relevant recognition element associated therewith.

A characteristic shared by the various embodiments mentioned of a method according to the invention is that said different sequences or different epitopes of immobilized biological or biochemical or synthetic recognition elements are selected from the group comprising nucleic acids (for example DNA, RNA, oligonucleotides) and nucleic acid analogs (e.g. PNA) as well as derivatives thereof with synthetic bases, monoclonal or polyclonal antibodies, peptides, enzymes, aptamers, synthetic peptide structures, glycopeptides, glycoproteins, oligosaccharides, lectins, soluble, membrane-bound proteins and proteins isolated from a membrane, such as receptors, ligands thereof, antigens for antibodies (e.g. biotin for streptavidin), “histidine-tag components” and complex-forming partners thereof, cavities generated by chemical synthesis for hosting molecular imprints, etc. It is also proposed that whole cells, cell components, cell membranes or fragments thereof are applied as biological or biochemical or synthetic recognition elements.

It is preferred that a referencing reagent required for certain embodiments of the method according to the invention comprises a label which is selected from among the group of, for example, luminescence labels, especially luminescent intercalators or “molecular beacons”, absorption labels, mass labels, especially metal colloids or plastic beads, spin labels, such as ESR or NMR labels, and radioactive labels.

It is preferred that said referencing reagent comprises a luminescence label or absorption label. In particular, said referencing reagent may also comprise an intercalator or a “molecular beacon”. It is preferred in this case that said intercalator or “molecular beacon” changes its signaling properties in the presence of the referencing reagent.

Before or during an analytical detection procedure, said referencing reagent may be cleaved off or remain associated with the recognition elements.

A further advantageous embodiment of the method according to the invention comprises the said referencing reagent including a component from among the group of, for example, polynucleotides, polynucleotides with synthetic bases, PNAs (“peptide nucleic acids”), PNAs with synthetic bases, proteins, antibodies, biotin, streptavidin, peptides, oligosaccharides, and lectins, etc.

A further characteristic shared by the mentioned embodiments of a method according to the invention is that the quantitative and/or qualitative detection of the said multitude of analytes comprises the use of one or more signaling components as labels, which may be selected from among the group comprising, for example, luminescence labels, especially luminescent intercalators or “molecular beacons”, absorption labels, mass labels, especially metal colloids or plastic beads, spin labels, such as ESR or NMR labels, and radioactive labels.

It is preferred that the label of the referencing reagent and/or an analyte detection optionally based on absorption and/or luminescence detection is based on the use of labels with the same or different absorption and/or luminescence wavelengths.

A special embodiment, based on the recognition elements immobilized in the measurement areas, in each case with an associated signaling component as label, comprises the said label also serving for analyte detection in addition to referencing the immobilization density of the recognition elements. For example, the said label may be a fluorescent intercalator which, bound to a single-stranded nucleic acid as immobilized recognition element, emits a very weak, but nevertheless measurable signal, from which the density of the recognition elements immobilized in the corresponding measurement areas can be determined. Upon hybridization with a (single-stranded) nucleic acid in an added sample as analyte, which is at least partly complementary, especially in the region of the immobilized intercalator, a marked increase may occur in the fluorescence intensity of this intercalator, on the basis of which the analyte concerned is then qualitatively and/or quantitatively detected on this measurement area.

The detection of analytes is preferably based on determining the change in one or more luminescences.

A possible embodiment comprises the excitation light from one or more light sources for generating the signals of signaling components for the purpose of chemical referencing and/or for the detection of one or more analytes being delivered in a epi-illumination configuration.

Other embodiments comprise the excitation light from one or more light sources for generating the signals of signaling components for the purpose of referencing the immobilization density and/or for the detection of one or more analytes being delivered in a transillumination configuration.

A preferred subject of the invention is an embodiment of the method according to the invention wherein the sensor platform is provided as an optical waveguide which is preferably essentially planar, and wherein the excitation light from one or more light sources is coupled into the optical waveguide using a method selected from the group formed by end-face (distal end) coupling, coupling via attached optical fibers as lightguides, prism coupling, grating coupling or evanescent coupling by overlapping of the evanescent field of said optical waveguide with the evanescent field of a further waveguide brought into near-field contact therewith.

It is preferred in this case if the in-coupling of the excitation light from one or more light sources into the optical waveguide is performed by means of an optical coupling element which is in contact therewith and which is selected from the group of optical fibers as lightguides, prisms, if necessary using an refractive index-matching liquid, and grating couplers.

Especially preferred is such an embodiment of the method according to the invention wherein the sensor platform comprises an optical thin-film waveguide with a layer (a) which is transparent for at least one excitation wavelength on a layer (b) which is likewise transparent for at least this excitation wavelength with a lower refractive index than layer (a), and wherein the excitation light from one or more light sources is coupled into layer (a) by means of one or more grating structures (c) modulated in layer (a).

This embodiment of the method may be carried out in such a manner that one or more liquid samples to be tested on said analytes are brought into contact with the measurement areas on the sensor platform and one or more luminescences generated in the near field of layer (a) from the measurement areas brought into contact with said sample or samples as a consequence of the binding of one or more analytes to the biological or biochemical or synthetic recognition elements immobilized in said measurement areas or of the interaction between said analytes and said immobilized recognition elements are measured, and additionally if necessary in locally resolved manner the available excitation light intensity in said measurement areas is referenced.

It is preferred if (1) the isotropically emitted luminescence or (2) luminescence that is in-coupled into the optically transparent layer (a) and out-coupled via grating structures (c) or luminescences of both parts (1) and (2) are measured at the same time.

Part of the method according to the invention is that, to generate luminescence, a luminescent dye or luminescent nanoparticle is used as luminescence label, which can be excited and emits at a wavelength between 300 nm and 1100 nm.

The luminescence label is preferably bound to the analyte or, in a competitive assay, to an analog of the analyte or, in a multistep assay, to one of the binding partners of the immobilized biological or biochemical or synthetic recognition elements or to the biological or biochemical or synthetic recognition elements.

Another embodiment of the method comprises the use of a second luminescence label or of further luminescence labels with excitation wavelengths either the same as or different from that of the first luminescence label and the same or different emission wavelength.

It is preferred here if the second luminescence label or further luminescence labels can be excited at the same wavelength as the first luminescence dye, but emit at different wavelengths.

In particular it is advantageous if the excitation spectra and emission spectra of the luminescence dyes used overlap only a little, if at all.

A variant of the method comprises using charge or optical energy transfer from a first luminescence dye serving as donor to a second luminescence dye serving as acceptor for the purpose of detecting the analyte.

Another possible embodiment of the method comprises determining the extent to which one or more luminescences are quenched.

A further embodiment of the method comprises determining changes in the effective refractive index on the measurement areas in addition to measuring one or more luminescences.

Characteristic for a further embodiment of the method is that the one or more luminescences and/or determinations of light signals at the excitation wavelength are measured polarization-selective.

It is preferred that the one or more luminescences are measured at a polarization that is different from the one of the excitation light.

A preferred embodiment of the method according to the invention comprises the density of the recognition elements immobilized in discrete measurement areas for the detection of different analytes on different measurement areas being selected in such a way that the luminescence signals upon determination of different analytes in a common array are of similar order of magnitude, i.e. that the related calibration curves for the analyte determinations to be performed at the same time can be recorded without a change in the settings of the electronic or opto-electronic system.

A further embodiment of the method comprises arrays of measurement areas being divided into segments of one or more measurement areas for determining analytes and regions between these measurement areas or additional measurement areas for the purpose of physical referencing, for example, of the excitation light intensity available in the measurement areas or of the influence of changes in external parameters, such as temperature, and also for the purpose of referencing of the influence of additional physicochemical parameters, such as nonspecific binding to the sensor platform of components of an applied sample. Nonspecific binding components of an applied sample may, for example, be the one or more analytes themselves, tracer reagents added to the sample for the detection of analyte, e.g. secondary, luminescently labeled antibodies in a sandwich immunoassay, or also parts of the sample matrix, especially if the sample medium is, for example, a body fluid and the sample has not undergone any further purification steps. For the determination of nonspecific binding, the areas intended for this purpose on the sensor platform may, for example, have been “passivated”, i.e. coated with a compound that is “chemically neutral” to the analyte, as described hereinbefore as a measure to reduce nonspecific binding.

For certain applications, for example for the detection of low-molecular-weight compounds in immunoanalysis or for the detection of single point mutations in nucleic acid analysis, it is hardly possible to exclude cross-reactivity with the (bio)chemically most similar cognates of the analyte concerned. For such applications, an advantageous embodiment of the method according to the invention is one in which one or more measurement areas of a segment or an array are assigned to determination of the same analyte and the immobilized biological or biochemical recognition elements thereof have differing degrees of affinity to said analyte. It is expedient in this case to select the recognition elements in such a manner that their affinities to different analytes which are (bio)chemically similar to one another change in different characteristic ways. The identity of the analyte can then be determined from the totality of the signals of different measurement areas with different recognition elements for an individual analyte, in a manner comparable to that for a fingerprint.

For other specific applications, in which the main focus concerns, for example, questions of the reproducibility of results using a large number of arrays on a common sensor platform, it is advantageous if two or more arrays have a similar geometric arrangement of measurement areas and/or segments of measurement areas for determining similar types of analyte on these arrays.

It can likewise be of advantage, especially for investigating the reproducibility of measurements on different measurement areas, if one or more arrays comprise segments of two or more measurement areas with similar biological or biochemical or synthetic recognition elements within the segment for analyte determination or referencing.

In other applications, it is essential to minimize the influences of systematic errors on the results, as may arise for example from a replication of similar structures on a common sensor platform. It may be of advantage in this case, for example, if two or more arrays have different geometric arrangements of measurement areas and/or segments of measurement areas for the determination of similar analytes on these arrays.

The method according to the invention using a kit according to the invention with a multitude of measurement areas in discrete arrays, of which many may in turn be arranged on a common sensor platform, offers the possibility of conducting many kinds of duplication or multiple performance of similar measurements using relatively small quantities of sample solutions, reagents or calibration solutions on one and the same platform under largely identical conditions. Thus, for example, statistical data can be generated in a single measurement which by conventional means would require a large number of individual measurements with a correspondingly longer total measurement time and consumption of greater amounts of samples and reagents. It is preferred if two or more identical measurement areas within a segment or an array are provided in each case for the determination of each analyte or for referencing. Said identical measurement areas may be arranged here, for example, in a continuous row or column or diagonal of an array or a segment of measurement areas. The aspects of referencing may be related to physical or physicochemical parameters of the sensor platform, such as local variations of the excitation light intensity (see also below), as well as effects of the sample, such as its pH, ionic strength, refractive index, temperature, etc.

For other applications, however, it may also be advantageous if said identical measurement areas are distributed statistically within an array or a segment of measurement areas.

As described in greater detail hereinbefore, a further essential aspect of the present invention comprises additional arrangements for locally resolved referencing of the excitation light intensity available in the measurement areas.

A possible embodiment of the method according to the invention thus comprises the locally resolved referencing of the excitation light intensity available in the measurement areas by means of simultaneous or sequential generation of an image of the light emanating from the sensor platform at the excitation wavelength. An image of the excitation light emanating from the sensor platform is preferably generated in this case via the same optical path as that used to record the luminescences emanating from the measurement areas.

Another possible embodiment of the method comprises the locally resolved referencing of the excitation light intensity available in the measurement areas by means of simultaneous or sequential generation of an image of the light emanating from the sensor platform at the luminescence wavelength.

A further embodiment comprises the arrangements for locally resolved referencing of the excitation light intensity available in the measurement areas being the simultaneous or sequential generation of an image of the light emanating from the sensor platform at an excitation wavelength other than that used for excitation of a luminescence.

The local resolution of the image for referencing of the excitation light emanating from the sensor platform is preferably lower than 100 μm on the sensor platform, and preferably lower than 20 μm.

An object of the method according to the invention comprises the locally resolved referencing of the excitation light intensity available in the measurement areas being performed by means of “luminescence marker spots”, i.e. determination of luminescence intensity from measurement areas with pre-immobilized luminescently labeled molecules (i.e. molecules which have already been deposited in these measurement areas before the application of a sample).

In this case, the “luminescence marker spots” are preferably applied in a pattern covering the whole sensor platform.

A further embodiment of the method according to the invention comprises the density of the luminescently labeled molecules being selected by mixture with similar types of unlabeled molecules during immobilization in such a manner that the luminescence intensity from the areas of luminescence marker spots is of similar order of magnitude as the luminescence intensity from the measurement areas intended for analyte detection.

A preferred embodiment of the method comprises the density and concentration of the luminescently labeled molecules within the “luminescence marker spots” in an array, preferably on the common sensor platform, being uniform.

It is further preferred if the locally resolved referencing of the excitation light intensity available in the measurement areas comprises the determination of an average of multiple locally resolved reference signals. In the case of nonstatistically, but systematically varying excitation light intensities in the form of a gradient over certain distances, interpolation on the expected value of excitation light intensity of a measurement area lying between different areas for locally resolved referencing may be advantageous.

The addition of one or more samples and of the tracer reagents to be used in the method of detection may take place sequentially in several steps. One or more samples are preferably incubated beforehand with a mixture of the various tracer reagents for determining the analytes to be detected in said samples and these mixtures then added in a single step to the related dedicated arrays on the sensor platform.

A preferred embodiment of the method according to the invention comprises the concentration of the detection reagents, such as secondary tracer antibodies and/or luminescence labels and optional additional luminescently labeled tracer reagents in a sandwich immunoassay, being selected in such a way that the luminescence signals upon the detection of different analytes in a common array are of the same order of magnitude, i.e. that the related calibration curves for the analyte determinations to be carried out simultaneously can be measured without a change in the settings of the opto-electronic system.

A further subject of an embodiment of the method according to the invention is the calibration of luminescences generated as a result of the binding of one or more analytes or as a result of the specific interaction with one or more analytes comprising the application of one or more calibration solutions with known concentrations of said analytes to be determined to the same or different measurement areas or segments of measurement areas or arrays of measurement areas on a sensor platform to which one or more of the samples to be tested are added in the same or in a separate step.

Characteristic for another preferred embodiment of the method is, that the calibration of luminescences generated as a result of the binding of one or more analytes or as the result of the specific interaction with one or more analytes comprises the comparison of the luminescence intensities after addition of an unknown sample and a control sample, such as a “wild type” DNA sample and a “mutant DNA” sample. It is possible here that the unknown sample and the control sample are added to different arrays.

Another variant of this method comprises adding the unknown sample and the control sample sequentially to the same array. In this embodiment, a regeneration step is generally necessary between addition of the unknown sample and the control sample, i.e. the dissociation of complexes of recognition element and analyte formed after addition of the first sample, followed by removal of the dissociated analyte molecules from the sample compartments, before the second sample can be added. In a similar manner, multiple samples may also be tested for their analytes in sequential form on an array of measurement areas.

Another possible embodiment of the method comprises the unknown sample and the control sample being mixed and then the mixture being added to one or more arrays of a sensor platform.

A further embodiment of the method according to the invention comprises the detection of the analytes to be determined in the unknown and the control sample being carried out using luminescence labels of different excitation and/or luminescence wavelengths for the unknown and the control sample.

For the determination of analytes from different groups, the detection is preferably carried out, for example, using two or more luminescence labels with differing excitation and/or luminescence wavelengths.

As described hereinbefore, the kit according to the invention, with its large number of measurement areas on a sensor platform, opens up the possibility of a simplified form of calibration for the qualitative and/or quantitative determination of one or more analytes on one or more arrays. In the best case, with this new form of calibrating the signals of a sensor platform according to the invention, it is only necessary to add a single calibration solution. In this further embodiment of the method according to the invention it is therefore preferred that several measurement areas with biological or biochemical or synthetic recognition elements immobilized there in differing controlled density are provided in one or more arrays for the determination of an analyte common to these measurement areas.

Characteristic for this further embodiment of the method is the possibility of establishing a calibration curve for an analyte with the application of just a single calibration solution when the concentration dependence of the binding signals between the analyte and its biological or biochemical or synthetic recognition elements is known and there is a sufficiently wide “variation” of these recognition elements immobilized in different controlled density in different measurement areas of an array.

Part of the invention is a method according to one of the embodiments mentioned hereinbefore for simultaneous or sequential, quantitative or qualitative determination of one or more analytes from the group of antibodies or antigens, receptors or ligands, chelators or “histidine tag components”, oligonucleotides, DNA or RNA strands, DNA or RNA analogs, enzymes, enzyme cofactors or inhibitors, lectins and carbohydrates.

Possible embodiments of the method comprise the samples to be tested being naturally occurring body fluids such as blood, serum, plasma, lymph or urine or egg yolk or optically turbid fluids or tissue fluids or surface water or soil or plant extracts or biological or synthetic process broths or being taken from biological tissue parts or from cell cultures or extracts.

A further subject of the invention is the use of a kit according to the invention and/or of an analytical system according to the invention and/or of a method according to the invention for quantitative or qualitative analysis for the determination of chemical, biochemical or biological analytes in screening methods in pharmaceutical research, combinatorial chemistry, clinical and preclinical development, for real-time binding studies and for the determination of kinetic parameters in affinity screening and in research, for qualitative and quantitative analyte determinations, especially for DNA- and RNA analysis, for the generation of toxicity studies and for the determination of gene and protein expression profiles, and for the determination of antibodies, antigens, pathogens or bacteria in pharmaceutical product development and research, human and veterinary diagnostics, agrochemical product development and research, for symptomatic and presymptomatic plant diagnostics, for patient stratification in pharmaceutical product development and for therapeutic drug selection, for the determination of pathogens, noxious substances and pathogens, especially salmonella, prions and bacteria, in food and environmental analysis.

The following examples explain the invention in more detail.

### EXAMPLE 1

1. Suitability of Nucleic Acids as Recognition Elements which are to be Immobilized, with a General Sequence of an Associated Signaling Component as Label for the Purpose of Referencing the Immobilization Density and Different Specific Sequences for Recognition and Binding of Different Analytes

For cloning, DNA fragments (inserts) of the organism to be studied are inserted into plasmid DNA (circular DNA sequences in bacteria or other microorganisms) using restriction endonucleases and ligases in order to produce so-called recombinant DNA.

Both the vectors (plasmids without incorporated foreign DNA) and also the DNA fragments to be replicated are “cut” in a defined and matching manner by means of endonucleases and spliced together by means of ligases. These vehicles are incorporated into bacterial host cells, in most cases E. coli cells, for example by electroporation. Using an antibiotic resistance procedure, the host cells selected from all cells subject to the method are those which have taken up the “vehicle” and are applied to and cultivated on a suitable solid culture medium or also cultivated in a liquid culture medium. The “vehicles” are replicated via natural growth of these bacteria cultures. In an analogous manner, linear DNA constructs, so-called bacteriophages and viruses which are able to infect bacterial cells, can be used as vehicles instead of circular plasmids.

The success of incorporating DNA fragments into a vector is tested using the ampicillin resistance method¹, and the success of incorporation into the bacterial cell is tested using the tetracycline resistance method². ¹ Bacteria into which DNA fragments are incorporated as “vehicles” are no longer resistant to this antibiotic. ² Vetors contain a gene for tetracycline resistance: if a recombinant DNA is incorporated into the bacterial cell, the cell is resistant to the antibiotic.

The bacterial cells which contain desired recombinant DNA are identified via replica plating and labeling using suitable radioactively labeled—complementary—nucleic acid probes. The recombinant DNA is isolated and purified using established methods: lysis of the cell wall, removal of cellular fragments by centrifugation, further purification by means of phenol extraction, and ethanol precipitation. Alternatively, commercially available DNA isolation kits may be used.

Instead of the process steps described hereinbefore, a cloning procedure may be applied using so-called “T vectors” [J. Sambrock and D. W. Russell, “Molecular Cloning—A Laboratory Manual”, Vol. 2 (2001), Section 8.35, Cold Spring Harbor Laboratory Press, Cold Spring Harbor, N.Y.].

It is characteristic of all recombinant DNA molecules prepared in this way that the base sequence of the vector is known. This gives rise to the possibility of using suitable endonucleases to excise from these plasmids pieces of DNA which contain the desired DNA fragment in the central area and DNA sequences of defined length and defined base sequence on the lateral margins.

Based on these DNA sequences with defined margin sections, two different methods were developed according to the invention for the purpose of referencing the density of recognition elements (“referencing of the immobilization density”) immobilized on a sensor platform as support: Every recognition element applied to a support surface carries one or two general DNA sequences, apart from the specific recognition region, which can be used for referencing purposes:

2.1. Preparation, Immobilization and Determination of the Immobilization Density of Nucleic Acids with an Associated Label as Signaling Component for the Purpose of Referencing the Immobilization Density

Recombinant DNA is prepared as plasmid or bacteriophage using the above procedures. By means of so-called polymerase chain reaction (PCR), selected DNA fragments are amplified by a factor of 10⁴ to 10⁶, using suitable so-called primers (oligonucleotides which serve as starting molecules for DNA polymerase). The base sequences of these primers are selected such that the amplified DNA fragments contain both the special originally imported DNA sequence and also parts of the vector sequences at the 3′ and 5′ end. These additional DNA sequences are typically about 10 to 40 bases in length.

With the variant presented in the first example for “referencing the immobilization density”, the so-called forward primer, reverse primer or both primers comprise—instead of native (nonmodified) nucleotides—such nucleotides which are derivatized at the nucleobase with fluorescent dyes such as Cy3 or Cy5. The labeling step is preferably carried out during oligonucleotide synthesis by the incorporation of fluorescently labeled uracil or cytosine. Primers with 15 to 25 base pairs each are typically used. The primers are selected such as to give an increase in length of preferably more than 5 nucleotides plus the length of the primer at the 5′ and the 3′ end of the original DNA incorporated into the vector. The polymerization reaction itself is carried out using commercially available Taq polymerase kits. Alternatively, fully synthetic recognition elements can be used with comparable properties. In a variation of this part of the method, nucleobases derivatized with a reactive group, for example an amino group, can also be used instead of nucleobases which are themselves derivatized with fluorescence dyes. In this case, the fluorescence dye to be used as fluorescence label is covalently bound in an additional step to the reactive group of the modified nucleotides only after the amplification is complete, and surplus fluorescence dye is then separated off.

The probe molecules are applied to the chemically activated surface of the support in the same way as the nonlabeled probe molecules, by means of mechanical pin or pen spotting techniques or application techniques analogous to inkjet spotting.

Since the sequence of the vector is known and the use of a uniform vector is generally desirable in cloning, it is possible to conduct the PCR reaction even of very different “inserts” using a primer pair. It is thus possible to obtain very uniform, reproducible and not least also very numerous and different probe molecules. By selecting fluorescence labels with suitable excitation and emission spectra (for example with emission of the fluorescence label for referencing the immobilization density, i.e. for determining the density of immobilized recognition elements in green-emitting light and an emission of the fluorescence label used for analyte detection in red-emitting light), “referencing of the immobilization density” can be conducted such that, after completion of hybridization, the detection can be carried out in commercial two-color scanners and the first color used for referencing and the second color for measuring the hybridization level. This method allows to determine in each measurement area the relative number of immobilized “probe DNA” as an immobilized recognition element. Based on this measurement, the fluorescence signals measured upon analyte detection can then be corrected (by dividing them by the corresponding reference signal), in order to obtain the relative binding signal, calculated with reference to the available recognition elements per measurement area. Since the fluorescence label used is covalently incorporated, no impairment of the hybridization capacity (resulting from steric hindrance) has to be expected. When the fluorescence labels are selected for “referencing the immobilization density” and for the analyte detection, it is generally preferred if the excitation and emission spectra of the different luminescence labels used only overlap very little, if at all.

2.2. Preparation, Immobilization and Determination of the Immobilization Density of Nucleic Acids with a General Molecular Sequence for the Purpose of Referencing the Immobilization Density

Recombinant DNA is prepared as plasmid or bacteriophage using the above procedures.

By means of so-called polymerase chain reaction (PCR), selected DNA fragments are amplified by a factor of 10⁴ to 10⁶, using suitable primers. The base sequences of these primers are selected such that the amplified DNA fragments contain both the special originally imported DNA sequence and also parts of the known vector sequences at the 3′ and 5′ end. These additional DNA sequences are typically about 10 to 40 bases in length.

Primers with 15 to 25 bases each are typically used. The primers are selected such as to give an increase in length of preferably more than 5 nucleotides plus the length of the primer at the 5′ and the 3′ end of the DNA originally incorporated into the vector. The polymerization reaction itself is carried out using commercially available Taq polymerase kits. Alternatively, fully synthetic recognition elements can be used with comparable properties.

The probe molecules are applied to the chemically activated surface of the substrate in the same way as the nonlabeled probe molecules, by means of mechanical pin or pen spotting techniques or application techniques analogous to ink-jet spotting.

In a hybridization step, fluorescently labeled oligonucleotide sequences, whose sequences are complementary to a general DNA part defined by the vector, are applied. As a consequence, a fluorescence intensity of each measurement area can be determined, the level of which corresponds essentially to the quantity of immobilized probe DNA. By means of dehybridization induced either thermally or by ionic strength, the resulting hybrid may—if necessary—be cleaved and the incorporated fluorescence flushed out with the probe.

This method is especially suitable for the representative quality control of production lots, because—apart from having to know the sequences of DNA regions originating from the plasmid vector—no information is needed about the recognition part of the DNA fragment and—if all recognition DNA has been prepared using the identical cloning technique—the measurement only requires one sort of nucleic acid probe.

2.3. Preparation and use of Synthetic Recognition Elements for a Kit According to the Invention and a Method According to the Invention for Analyte Detection

Alternatively, shorter polymer sequences (or oligonucleotide sequences) (<100 bases) may be synthetically produced under low-cost conditions. It is possible to create polynucleotides out of two separate building blocks—a general sequence and a specific sequence suitable for the recognition of individual expressed genes.

The bases of the general sequence in this case may consist of native nucleobases or either partly or wholly of fluorescently labeled bases. Depending on the nature of these building blocks, the recognition elements may be used in a manner analogous to methods 2.1. and 2.2.

### EXAMPLE 3

Kit According to the Invention with Immobilized Antibodies with an Associated Fluorescence Label as Signaling Component for the Purpose of Referencing the Immobilization Density and a Method According to the Invention for Analyte Detection

3.1. Sensor Platform

A sensor platform is used with the external dimensions of 57 mm in width (parallel to the grating lines of a grating structure (c) modulated in layer (a) of the sensor platform)×14 mm in length (perpendicular to the grating structure)×0.7 mm in height, on the surface of which 6 microflow cells can be created in the pattern of part of a column of a standard microtiter plate (9 mm spacing) by combination with a polycarbonate plate featuring open cavities in the direction of the sensor platform with the internal dimensions of 5 mm wide×7 mm long×0.15 mm high. The polycarbonate plate may be adhered to the sensor platform in such a way that the cavities are then tightly sealed against each other. This polycarbonate plate is constructed such that it can be joined together with a carrier (“meta-carrier”) with the basic dimensions of standard microtiter plates in such a way that the pitch (arrangement of rows or columns) of the inlets of the flow cells matches the pitch of the wells of a standard microtiter plate.

The substrate material (optically transparent layer (b) comprises AF 45 glass (refractive index n=1.52 at 633 nm). The substrate features a pair of in-coupling and out-coupling gratings with grating lines (318 nm period) running parallel with the width of the sensor platform at a grating depth of 12±3 nm, wherein the grating lines are drawn over the whole width of the sensor platform. The distance between the two consecutive gratings is 9 mm, and the length of the individual grating structures (parallel with the length of the sensor platform) is 0.5 mm. The distance between the in-coupling and out-coupling grating of a grating pair is selected such that the excitation light in each case can be in-coupled within the region of the sample compartment, after combination of the sensor platform with the aforementioned polycarbonate plate, whereas the out-coupling takes place outside the region of the sample compartment. The waveguiding, optically transparent layer (a) consisting of Ta₂O₅ on the optically transparent layer (b) has a refractive index of 2.15 at 633 nm (layer thickness 150 nm).

The sample compartments formed from the sensor platform and the polycarbonate plate combined therewith feature conical openings bored out on the demarcation areas opposite to the sensor platform, so that the sample compartments can be filled or emptied by pressing in standardized, commercially available polypropylene pipette tips.

To prepare for immobilization of the biochemical or biological or synthetic recognition elements, the sensor platform is cleaned first with isopropanol, then with concentrated H₂SO₄, 2.5% ammonium peroxodisulfate in a sonication device and then incubated for 2 hours at room temperature with 0.5 mM dodecyl monophosphate (ammonium salt), while the solution is constantly stirred. In this process, a homogeneous, hydrophobic surface forms by means of self-assembly.

3.2. Preparation and Immobilization of Antibodies with an Associated Fluorescence Label as Signaling Component for the Purpose of Referencing the Immobilization Density

Monoclonal antibodies (against 8 interleukins IL#1 to IL#8 in the concrete example) are fluorescently labeled with Cy3 using a standard technique. In each case, the antibody to be labeled is dissolved in 0.1 M carbonate buffer pH 9.2 at a concentration of about 1 mg/ml, so that the primary amines of e.g. lysine side chains of the protein are present in a completely deprotonated state. Part of a Cy3-NHS ester dissolved beforehand in DMSO is added to this solution and incubated for one hour in the dark at room temperature with gentle stirring. The concentration of DMSO in the total solution must not be higher than 1% in order to avoid a denaturation and thus a loss of function of the antibody to be labeled. After completion of the reaction, in which a covalent bond is established between the fluorescence label (Cy3) and the lysine side chains of the protein, that part of the dye which has not reacted with the protein is chromatographically separated. In a variation of the method according to the invention or the kit according to the invention, the molar ratio of antibodies and fluorescence labels (Cy3) during the reaction is selected such that not every antibody molecule but, for example, only about one of ten antibody molecules is covalently labeled. The ratio of Cy3-labeled to unlabeled antibodies may, according to current methods, be verified on the basis of the absorption spectrum.

In a concentration of 50-150 μg/ml in phosphate-buffered salt solution (pH 7.4), comprising in addition suitable additives for preserving the functionality of the immobilized proteins, the fluorescence-labeled 8 different (primary) antibodies against interleukins IL#1 to IL#8 (or mixtures of fluorescently labeled and unlabeled antibodies against said interleukins) are applied by means of an ink-jet spotter and dried. The mean diameter of the spots, with a (center-to-center) distance of 0.35 mm, is 0.15 mm. Eight different antibodies for the recognition of cytokines, in particular of different interleukins, are used in 20 rows each of a single array with a total of 160 spots. To obtain data for statistical assay reproducibility at the same time from each individual measurement per sample to be applied, 20 measurement areas are created per array with the same interleukin antibodies as biological recognition elements.

Six such arrays of identical geometry are created on the sensor platform in a 9 mm pitch (arranged in a column).

To the sensor platform thus prepared, the polycarbonate plate described hereinbefore is applied in such a way that the individual sample compartments feature a tight fluidic seal against each other and the protein microarrays created are located each within one of the 6 sample compartments with the corresponding in-coupling grating (c).

3.3. Performance of a Multianalyte Immunoassay for the Determination of 8 Cytokines Referenced for the Surface Density of the Immobilized Recognition Elements

The format of a sandwich assay is selected for the specific recognition of the cytokines to be detected. For the selected cytokines (interleukins IL#1 to Il#8), 6 calibration solutions are prepared comprising each of the 8 interleukins in identical concentration in PBS buffer pH 7.4 with the addition of 0.1% serum albumin and 0.05% Tween 20 (interleukin concentrations 0, 50, 125, 250, 500,1000 pg/ml). The individual concentration solutions are then pre-incubated at 37° C. for one hour with a mixture comprising the corresponding (8 different) specific biotinylated secondary anti-interleukin antibodies (in each case 1-2 nanomolar) and Cy5-labeled streptavidin (5-15 nM). Then 50 μl each of the 6 individual calibration solutions is filled into each of the 6 flow cells on the sensor platform and incubated for a further 2 hours at 37° C. with the respective array on the sensor platform, so that the complexes formed in the pre-incubation step from the respective interleukins, specific secondary, biotinylated anti-interleukin antibodies and Cy-5-labeled streptavidin can bind to the primary anti-interleukin antibodies immobilized in the discrete measurement areas (spots).

After completion of the binding step, the flow cells are washed with buffer (phosphate-buffered salt solution with addition of 0.1% serum albumin and 0.05% Tween 20).

The sensor platform with the adjoined polycarbonate plate is then inserted into a “meta-carrier”, as described hereinbefore (Example 3.1), and—after a further 15-minute incubation period (for equilibration at room temperature) in buffer—is inserted into an analytical system according to the invention and measured.

Through selection of fluorescence labels with suitable excitation and emission spectra (for example with an emission of the fluorescence label for referencing the immobilization density, i.e. for determining the density of immobilized recognition elements in green-emitting light (e.g. Cy3), and an emission of the fluorescence label used for analyte detection in red-emitting light (e.g. Cy5)), the “referencing of immobilization density” can be carried out in such a way that, after completion of the assay, the detection takes place in an analytical system according to the invention, for example using commercial two-color scanners or also an optical system, as described in PCT/EP 01/10012, and the first color can be used for referencing and the second color for measuring the specific assay signal. This method allows the relative number of immobilized antibodies as immobilized recognition elements to be determined in every measurement area. Based on this measurement, the fluorescence signals measured upon the detection of analyte can then be corrected (by dividing them by the corresponding reference signal), in order to obtain the relative binding signal, calculated with reference to the available recognition elements per measurement area. Since the fluorescence label used for referencing of the immobilization density is covalently incorporated, no compromise of functionality (as a result of steric hindrance caused by the fluorescence label), i.e. of the capacity of the fluorescence-labeled immobilized antibody for specific recognition and binding of the antigen, has to be expected. When the fluorescence labels are selected for “referencing the immobilization density” and for the analyte detection, it is generally preferred if the excitation and emission spectra of the different luminescence labels used only overlap very little, if at all.

What is claimed is:

1. A kit for simultaneous qualitative and/or quantitative detection of a multitude of analytes, comprising
a sensor platform
at least one array of biological or biochemical or synthetic recognition elements immobilized in discrete measurement areas (d) directly or by means of an adhesion-promoting layer on the sensor platform for specific recognition and/or binding of said analytes and/or for specific interaction with said analytes,
wherein for purposes of “referencing the immobilization density”, i.e. for locally resolved determination of the density of immobilized recognition elements in the measurement areas, these recognition elements are associated in each case with a signaling component as label and/or said biological or biochemical or synthetic recognition elements comprise a certain molecular sequence or a certain molecular epitope or a certain molecular recognition group, to which a tracer reagent (referencing reagent), if necessary using a signaling component associated therewith as label, binds for determination of the said density of immobilized recognition elements.

2. A kit according to claim 1, comprising the immobilized recognition elements in the measurement areas each comprising a general molecular sequence or a general epitope or general molecular recognition group for the purpose of referencing the immobilization density and one or more different sequences or different epitopes or different molecular recognition groups for the recognition and/or binding of different analytes.

3. A kit according to any of claims 1-2, comprising, for the said purpose of referencing the immobilization density, a referencing reagent for recognition and/or binding to said general sequence or to said general epitope or to said general molecular recognition group of biological or biochemical or synthetic recognition elements immobilized in the same measurement area on the sensor platform being co-immobilized, if necessary in association with said immobilized recognition elements.

4. A kit according to any of claims 1-2, comprising, for the said purpose of referencing the immobilization density, a referencing reagent for recognition and/or binding to said general sequence or to said general epitope or to said general molecular recognition group of immobilized biological or biochemical or synthetic recognition elements on the sensor platform being applied to the measurement areas of the sensor platform after immobilization of the biological or biochemical or synthetic recognition elements.

5. A kit according to any of claims 1-2, comprising, for the said purpose of referencing the immobilization density, as part of a quality control during or after manufacture of the sensor platform, a referencing reagent for recognition and/or binding to said general sequence or to said general epitope or to said general molecular recognition group of immobilized biological or biochemical or synthetic recognition elements on the sensor platform being applied to the measurement areas of the sensor platform after immobilization of the biological or biochemical or synthetic recognition elements.

6. A kit according to any of claims 1-2, comprising, for said purpose of referencing the immobilization density, a referencing reagent for recognition and/or binding to said general sequence or to said general epitope or to said general molecular recognition group of the immobilized biological or biochemical or synthetic recognition elements on the sensor platform being applied to the measurement areas of the sensor platform in the course of a detection procedure for the determination of one or more analytes.

7. A kit according to any of claims 1-6, comprising said general molecular sequence or said general epitope or said general molecular recognition group (such as biotin) of the immobilized biological or biochemical or synthetic recognition elements being selected from the group that is formed by polynucleotides, polynucleotides with synthetic bases, PNAs (“peptide nucleic acids”), PNAs with synthetic bases, proteins, antibodies, peptides, oligosaccharides, lectins, etc.

8. A kit according to claim 7, comprising said general sequence of immobilized biological or biochemical or synthetic recognition elements having a length of 5-500, preferably of 10-100 bases.

9. A kit according to claim 1, comprising the immobilized recognition elements in the measurement areas in each case being associated with a signaling component as label.

10. A kit according to claim 9, comprising said signaling component as label changing its signaling properties upon the binding of an analyte to the respective recognition element associated therewith.

11. A kit according to any of claims 1-10, comprising said different sequences or different epitopes or different molecular recognition groups of immobilized biological or biochemical or synthetic recognition elements being selected from the group comprising nucleic acids (for example DNA, RNA, oligonucleotides) and nucleic acid analogs (e.g. PNA) as well as derivatives thereof with synthetic bases, monoclonal or polyclonal antibodies, peptides, enzymes, aptamers, synthetic peptide structures, glycopeptides, glycoproteins, oligosaccharides, lectins, soluble, membrane-bound proteins and proteins isolated from a membrane, such as receptors, ligands thereof, antigens for antibodies (e.g. biotin for streptavidin), “histidine-tag components” and complex-forming partners thereof, cavities generated by chemical synthesis for hosting molecular imprints, etc.

12. A kit according to any of claims 1-11, comprising said referencing reagent including a label which is selected from among the group of, for example, luminescence labels, especially luminescent intercalators or “molecular beacons”, absorption labels, mass labels, especially metal colloids or plastic beads, spin labels, such as ESR or NMR labels, and radioactive labels.

13. A kit according to any of claims 1-12, comprising said referencing reagent including a luminescence label or absorption label.

14. A kit according to any of claims 1-12, comprising said referencing reagent including an intercalator or a “molecular beacon”.

15. A kit according to claim 14, comprising said referencing reagent including an intercalator or a “molecular beacon” which changes its signaling properties in the presence of the referencing reagent.

16. A kit according to any of claims 1-15, comprising said referencing reagent being cleaved off before or during the analyte detection procedure or remaining associated with the recognition elements.

17. A kit according to any of claims 1-16, comprising said referencing reagent including a component from among the group that is formed by polynucleotides, polynucleotides with synthetic bases, PNAs (“peptide nucleic acids”), PNAs with synthetic bases, proteins, antibodies, biotin, streptavidin, peptides, oligosaccharides, lectins, etc.

18. A kit according to any of claims 1-17, comprising the quantitative and/or qualitative detection of the said multitude of analytes including the use of one or more signaling components as labels, which may be selected from among the group that is formed by, for example, luminescence labels, especially luminescent intercalators or “molecular beacons”, absorption labels, mass labels, especially metal colloids or plastic beads, spin labels, such as ESR or NMR labels, and radioactive labels.

19. A kit according to any of claims 12-18, comprising the label of the referencing reagent and/or an analyte detection optionally based on absorption and/or luminescence detection being based on the use of labels with the same or different absorption and/or luminescence wavelengths.

20. A kit according to any of claims 1-19 and claim 9, comprising said label also serving for analyte detection in addition to referencing the immobilization density of the recognition elements.

21. A kit according to any of claims 1-20, comprising the analyte detection being based on a determination of the change in one or more luminescences.

22. A kit according to one of claims 12-21, comprising the excitation light from one or more light sources for generating the signals of signaling components for the purpose of referencing the immobilization density and/or for the detection of one or more analytes being delivered in an epi-illumination configuration.

23. A kit according to any of claims 1-22, wherein the sensor platform material which is in contact with the measurement areas is transparent or absorbent for at least one excitation wavelength within a depth of at least 200 nm from the measurement areas.

24. A kit according to any of claims 12-21, comprising the excitation light from one or more light sources for generating the signals of signaling components for the purpose of referencing the immobilization density and/or for the detection of one or more analytes being delivered in a transillumination configuration.

25. A kit according to any of claims 1-24, comprising the sensor platform material being transparent for at least one excitation wavelength.

26. A kit according to any of claims 12-25, comprising the sensor platform being provided as an optical waveguide which is preferably essentially planar.

27. A kit according to claim 26, wherein the sensor platform comprises an optically transparent material from the group that is formed by silicates, e.g. glass or quartz, transparent thermoplastic or moldable plastic, for example polycarbonate, polyimide, acrylates, especially polymethylmethacrylate, or polystyrenes.

28. A kit according to claim 27, wherein the sensor platform comprises an optical thin-film waveguide with a layer (a) which is transparent for at least one excitation wavelength on a layer (b) which is likewise transparent for at least this excitation wavelength with a lower refractive index than layer (a).

29. A kit according to any of claims 26-28, wherein the excitation light from one or more light sources is coupled into the optical waveguide using a method selected from the group formed by end-face (distal end) coupling, coupling via attached optical fibers as light guides, prism coupling, grating coupling or evanescent coupling by overlapping of the evanescent field of said optical waveguide with the evanescent field of a further waveguide brought into near-field contact therewith.

30. A kit according to any of claims 26-28, wherein the in-coupling of the excitation light from one or more light sources into the optical waveguide is performed by means of an optical coupling element which is in contact therewith and which is selected from the group of optical fibers as lightguides, prisms, if necessary using a refractive index-matching liquid, and grating couplers.

31. A kit according to claim 30, comprising the excitation light from one or more light sources being coupled into layer (a) by means of one or more grating structures (c) modulated in layer (a).

32. A kit according to claim 31, wherein the sensor platform comprises uniform, non-modulated areas of layer (a), which are preferably arranged in the direction of propagation of the excitation light in-coupled into layer (a) via a grating structure (c) and guided in layer (a).

33. A kit according to any of claims 31-32, comprising grating structures (c) serving for the in-coupling of excitation light towards the measurement areas (d) and/or for the out-coupling of luminescence light back-coupled into layer (a)

34. A kit according to any of claims 31-33, comprising the sensor platform including numerous grating structures (c) of similar or different periods, with optionally adjacent uniform, nonmodulated regions of layer (a) on a common, continuous substrate.

35. A kit according to any of claims 31-34, wherein a dedicated grating structure (c) for out-coupling of the guided excitation light is provided following, in direction of propagation of the in-coupled excitation light, subsequent to each array of measurement areas, wherein, perpendicular to the direction of propagation of the in-coupled excitation light, individual grating structures for different arrays can be provided, or these grating structures can also extend in this direction (perpendicular to the direction of propagation of the in-coupled excitation light) over the whole sensor platform.

36. A kit according to any of claims 31-35, wherein the sensor platform comprises a superposition of two or more grating structures of different periodicities for the in-coupling of excitation light of different wavelengths, the grating lines being parallel or not parallel, preferably not parallel, to each other, wherein in the case of two superimposed grating structures their grating lines are preferably perpendicular to each other.

37. A kit according to any of claims 31-36, comprising a grating structure (c) or a superposition of several grating structures in layer (a) being essentially modulated across the whole area of the sensor platform.

38. A kit according to any of claims 31-37, comprising the sensor platform being furnished with optically or mechanically recognizable markings to facilitate adjustment in an optical system and/or for connection to sample compartments as part of an analytical system.

39. A kit according to any of claims 28-38, wherein an additional optically transparent layer (b′) with a lower refractive index than that of layer (a) and with a thickness of 5 nm-10,000 nm, preferably 10 nm-1000 nm, is located between the optically transparent layers (a) and (b) and in contact with layer (a).

40. A kit according to any of claims 1-39, wherein an adhesion-promoting layer (f), preferably with a thickness of less than 200 nm, more preferably of less than 20 nm, is deposited on the optically transparent layer (a), for the immobilization of the biological or biochemical or synthetic recognition elements in the discrete measurement areas, and wherein the adhesion-promoting layer (f) preferably comprises a chemical compound from the groups comprising silanes, epoxides, functionalized, charged or polar polymers, and “self-organized passive or functionalized monolayers or multiple layers”.

41. A kit according to any of claims 1-40, wherein laterally separated measurement areas (d) are generated by laterally selective deposition of biological or biochemical or synthetic recognition elements on the sensor platform, preferably using a method of the group of methods comprising ink jet spotting, mechanical spotting using pen, pin or capillary, “micro contact printing”, fluidic contacting of the measurement areas with the biological or biochemical or synthetic recognition elements upon their supply in parallel or crossed micro channels, upon application of pressure differences or electric or electromagnetic potentials, and photochemical or photolithographic immobilization methods.

42. A kit according to any of claims 1-41, comprising the density of the recognition elements immobilized in discrete measurement areas for the detection of different analytes on different measurement areas being selected in such a way that the signals upon determination of different analytes in a common array are of similar order of magnitude, i.e. that, if necessary, the related calibration curves for the analyte determinations to be performed at the same time may be recorded without a change in the settings of the electronic or opto-electronic system.

43. A kit according to any of claims 1-42, comprising arrays of measurement areas being divided into segments of one or more measurement areas for the determination of analytes and regions between these measurement areas or additional measurement areas for the purpose of the physical referencing, for example, of the excitation light intensity available in the measurement areas or of the influence of changes in external parameters, such as temperature, and for the purpose of referencing the influence of additional physicochemical parameters, such as nonspecific binding of components of an applied sample to the sensor platform.

44. A kit according to any of claims 1-43, wherein regions between the discrete measurement areas (d) are “passivated” in order to minimize nonspecific binding of analytes or their tracer compounds, i.e. that compounds are deposited between the discrete measurement areas (d) which are “chemically neutral” to the analyte, preferably for example compounds from groups comprising albumins, especially bovine serum albumin or human serum albumin, casein, nonspecific polyclonal or monoclonal, heterologous or empirically nonspecific antibodies for the analyte or analytes to be determined (especially for immunoassays), detergents (such as Tween20®), fragmented natural or synthetic DNA not hybridizing with polynucleotides to be analyzed, such as extract from herring or salmon sperm (especially for polynucleotide hybridization assays), or also uncharged but hydrophilic polymers, such as polyethylene glycols or dextrans.

45. A kit according to any of claims 1-44, comprising up to 100,000 measurement areas being provided in a 2-dimensional arrangement and a single measurement area occupying an area of 0.001 mm²-6 mm².

46. A kit according to any of claims 1-45, comprising the upper surface of the sensor platform, with the measurement areas generated thereon, being combined with a further body over the optically transparent layer (a) in such a way that one or more cavities are formed between the sensor platform as baseplate and said body for the generation of one or more sample compartments which are fluidically sealed against one another and each of which comprises one or more measurement areas or segments or arrays of measurement areas.

47. A kit with an arrangement of sample compartments according to claim 46, comprising the sample compartments as flow cells fluidically sealed against one another being formed in each case with at least one inlet and at least one outlet and optionally at least one outlet of each flow cell in addition leading to a reservoir fluidically connected to this flow cell to receive fluid exiting the flow cell.

48. A kit according to any of claims 1-46, comprising sample compartments being open on that side of the body combined with the sensor platform as baseplate which lies opposite the measurement areas.

49. A kit according to any of claims 46-48, wherein the arrangement of sample compartments comprises 2-2000, preferably 2-400, especially preferably 2-100 sample compartments.

50. A kit according to any of claims 46-49, comprising the pitch (geometric arrangement in rows and/or columns) of the sample compartments matching the pitch of the wells on a standard microtiter plate.

51. A kit according to any of claims 22-50, wherein additional means are provided for locally resolved referencing of the excitation light intensity available in the measurement areas.

52. A kit according to claim 51, wherein the means for locally resolved referencing of the excitation light intensity available in the measurement areas comprise the simultaneous or sequential generation of an image of the light at the excitation wavelength emanating from the sensor platform.

53. A kit according to any of claims 51-52, wherein the means for locally resolved referencing of the excitation light intensity available in the measurement areas comprise the determination of the background signals at the respective luminescence wavelength adjacent to or between the measurement areas.

54. A kit according to any of claims 51-53, comprising the locally resolved referencing of the excitation light intensity available in the measurement areas being performed by means of “luminescence marker spots”, i.e. determination of luminescence intensity from measurement areas with pre-immobilized luminescently labeled molecules (i.e. molecules have already been deposited in these measurement areas before application of a sample).

55. A kit according to any of claims 1-54, wherein additionally means for the calibration of luminescences resulting from the binding of one or more analytes or from the specific interaction with one or more analytes comprise the application of calibration solutions with known concentrations of the analytes to be detected on a predetermined number of arrays.

56. A kit according to any of claims 1-55, comprising several measurement areas with biological or biochemical or synthetic recognition elements immobilized there in differing controlled density being provided in one or more arrays for the determination of an analyte common to these measurement areas.

57. A kit according to claim 56, comprising the possibility of establishing a calibration curve for an analyte with the application of just a single calibration solution when the concentration dependence of the signals for the binding between the analyte and its biological or biochemical or synthetic recognition elements is known and there is a sufficiently wide “variation” of these recognition elements immobilized in different controlled density in different measurement areas of an array.

58. Use of a kit according to any of claims 1-57 in an analytical system for the determination of one or more luminescences.

59. A method for simultaneous qualitative and/or quantitative detection of a multitude of analytes using a kit according to any of claims 1-57, comprising, for the purpose of “referencing the immobilization density”, i.e. for locally resolved determination of the density of immobilized biological or biochemical or synthetic recognition elements in the measurement areas, these recognition elements being associated in each case with a signaling component as label and/or said biological or biochemical or synthetic recognition elements comprise a certain molecular sequence or a certain molecular epitope or a certain molecular recognition group, to which a tracer reagent (referencing reagent), if necessary using a signaling component associated therewith as label, binds and the signals of said signaling components being recorded in a locally resolved manner.

60. A method according to claim 59, comprising the determination of the immobilization density of the biological or biochemical or synthetic recognition elements on the sensor platform and the detection of said multitude of analytes being carried out independently of each other.

61. A method according to claim 60, comprising the determination of the immobilization density of the biological or biochemical or synthetic recognition elements on the sensor platform being carried out as part of the quality control during or after the manufacture of said sensor platform.

62. A method according to any of claims 59-61, wherein the immobilized recognition elements in the measurement areas each comprise a general molecular sequence or a general epitope or a general molecular recognition group for the purpose of referencing the immobilization density and a different sequence or different epitope or different molecular recognition group for the recognition and/or binding of different analytes.

63. A method according to any of claims 59-61, comprising for the said purpose of referencing the immobilization density, a referencing reagent for recognition and/or binding to said general sequence or to said general epitope or to said general molecular recognition group of the biological or biochemical or synthetic recognition elements immobilized in the same measurement area on the sensor platform being co-immobilized, if necessary in association with said immobilized recognition elements.

64. A method according to any of claims 59-62, comprising for the said purpose of referencing the immobilization density, a referencing reagent for recognition and/or binding to said general sequence or to said general epitope or to said general molecular recognition group of the immobilized biological or biochemical or synthetic recognition elements on the sensor platform being applied to the measurement areas of the sensor platform after immobilization of the biological or biochemical or synthetic recognition elements.

65. A method according to any of claims 59-62, comprising, for the said purpose of referencing the immobilization density, as part of a quality control during or after manufacture of the sensor platform, a referencing reagent for recognition and/or binding to said general sequence or to said general epitope or to said general molecular recognition group of immobilized biological or biochemical or synthetic recognition elements on the sensor platform being applied to the measurement areas of the sensor platform after immobilization of the biological or biochemical or synthetic recognition elements.

66. A method according to any of claims 59-62, comprising, for said purpose of referencing the immobilization density, a referencing reagent for recognition and/or binding to said general sequence or to said general epitope or to said general molecular recognition group of the immobilized biological or biochemical or synthetic recognition elements on the sensor platform being applied to the measurement areas of the sensor platform in the course of a detection procedure for the determination of one or more analytes.

67. A method according to any of claims 63-66, comprising said general molecular sequence or said general epitope or said general molecular recognition group (such as biotin) of the immobilized biological or biochemical or synthetic recognition elements being selected from the group that is formed by polynucleotides, polynucleotides with synthetic bases, PNAs (“peptide nucleic acids”), PNAs with synthetic bases, proteins, antibodies, peptides, oligosaccharides, lectins, etc.

68. A method according to claim 67, comprising said general sequence of immobilized biological or biochemical or synthetic recognition elements having a length of 5-500, preferably 10-100 bases.

69. A method according to any of claims 59-68, comprising the immobilized recognition elements in the measurement areas in each case being associated with a signaling component as label.

70. A method according to claim 69, comprising said signaling component as label changing its signaling properties upon the binding of an analyte to the respective recognition element associated therewith.

71. A method according to any of claims 59-70, comprising said different sequences of immobilized biological or biochemical or synthetic recognition elements being selected from the group that is formed by nucleic acids (for example DNA, RNA, oligonucleotides) and nucleic acid analogs (e.g. PNA) as well as derivatives thereof with synthetic bases, monoclonal or polyclonal antibodies, peptides, enzymes, aptamers, synthetic peptide structures, glycopeptides, glycoproteins, oligosaccharides, lectins, soluble, membrane-bound proteins and proteins isolated from a membrane, such as receptors, ligands thereof, antigens for antibodies, “histidine-tag components” and complex-forming partners thereof, cavities generated by chemical synthesis for hosting molecular imprints, etc.

72. A method according to any of claims 59-71, wherein the determination of the immobilization density of the biological or biochemical or synthetic recognition elements comprises the locally resolved determination of the signals of a signaling component as label, as a part of said referencing reagent, which is selected from among the group that is formed, for example, by luminescence labels, especially luminescent intercalators or “molecular beacons”, absorption labels, mass labels, especially metal colloids or plastic beads, spin labels, such as ESR or NMR labels, and radioactive labels.

73. A method according to any of claims 59-72, comprising said referencing reagent including a luminescence label or absorption label.

74. A method according to any of claims 59-72, comprising said referencing reagent including an intercalator or a “molecular beacon”.

75. A method according to any of claims 59-74, comprising in each case a label being associated with the biological or biochemical or synthetic recognition elements immobilized in discrete measurement areas (d), wherein this component is preferably an intercalator or a “molecular beacon” which changes its signaling properties in the presence of the referencing reagent.

76. A method according to any of claims 59-75, comprising said referencing reagent being cleaved off before or during the analyte detection procedure or remaining associated with the recognition elements.

77. A method according to any of claims 59-76, wherein said referencing reagent comprises a component from among the group that is formed by polynucleotides, polynucleotides with synthetic bases, PNAs (“peptide nucleic acids”), PNAs with synthetic bases, proteins, antibodies, biotin, streptavidin, peptides, oligosaccharides, lectins, etc.

78. A method according to one of claims 59-77, comprising the quantitative and/or qualitative detection of the said multitude of analytes including the use of one or more signaling components as labels, which may be selected from among the group formed by, for example, luminescence labels, especially luminescent intercalators or “molecular beacons”, absorption labels, mass labels, especially metal colloids or plastic beads, spin labels, such as ESR or NMR labels, and radioactive labels.

79. A method according to any of claims 72-78, comprising the label of the referencing reagent and/or an analyte detection optionally based on absorption and/or luminescence detection being based on the use of labels with the same or different absorption and/or luminescence wavelengths.

80. A method according to any of claims 59-79 and claim 74, comprising said label also serving for analyte detection in addition to referencing the immobilization density of the recognition elements.

81. A method according to any of claims 59-80, comprising the analyte detection being based on a determination of the change in one or more luminescences.

82. A method according to any of claims 72-81, comprising the excitation light from one or more light sources for generating the signals of signaling components for the purpose of referencing the immobilization density and/or for the detection of one or more analytes being delivered in an epi-illumination configuration.

83. A method according to any of claims 72-81, comprising the excitation light from one or more light sources for generating the signals of signaling components for the purpose of referencing the immobilization density and/or for the detection of one or more analytes being delivered in a transillumination configuration.

84. A method according to any of claims 81-83, wherein the sensor platform is provided as an optical waveguide which is preferably essentially planar, and wherein the excitation light from one or more light sources is coupled into the optical waveguide using a method selected from the group formed by end-face (distal end) coupling, coupling via attached optical fibers as lightguides, prism coupling, grating coupling or evanescent coupling by overlapping of the evanescent field of said optical waveguide with the evanescent field of a further waveguide brought into near-field contact therewith.

85. A method according to claim 84, wherein the in-coupling of the excitation light from one or more light sources into the optical waveguide is performed by means of an optical coupling element which is in contact therewith and which is selected from the group of optical fibers as lightguides, prisms, if necessary using an refractive index-matching liquid, and grating couplers.

86. A method according to claim 84, wherein the sensor platform comprises an optical thin-film waveguide with a layer (a) which is transparent for at least one excitation wavelength on a layer (b) which is likewise transparent for at least this excitation wavelength with a lower refractive index than layer (a), and wherein the excitation light from one or more light sources is coupled into layer (a) by means of one or more grating structures (c) modulated in layer (a).

87. A method according to claim 86, comprising one or more liquid samples to be tested for said analytes being brought into contact with the measurement areas on the sensor platform, one or more luminescences generated in the near field of the layer (a) from measurement areas brought into contact with said sample or samples, as a result of the binding of one or more analytes to the biological or biochemical or synthetic recognition elements immobilized in said measurement areas or as a result of the interaction between said analytes and said immobilized recognition elements, being measured and if necessary the excitation light intensity available in said measurement areas being additionally referenced in a locally resolved manner.

88. A method according to any of claims 86-87, wherein (1) the isotropically emitted luminescence or (2) luminescence that is in-coupled into the optically transparent layer (a) and out-coupled via grating structures (c) or luminescences of both parts (1) and (2) are measured at the same time.

89. A method according to any of claims 81-86, comprising—for the generation of luminescence—the use of a luminescent dye or of a luminescent nanoparticle as luminescence label which can be excited and emits at a wavelength between 300 nm and 1100 nm.

90. A method according to claim 89, comprising the luminescence label being bound to the analyte or, in a competitive assay, to an analog of the analyte or, in a multistep assay, to one of the binding partners of the immobilized biological or biochemical or synthetic recognition elements or to the biological or biochemical or synthetic recognition elements.

91. A method according to any of claims 89-90, comprising the use of a second luminescence label or of further luminescence labels with excitation wavelengths either the same as or different from that of the first luminescence label and the same or different emission wavelength.

92. A method according to any of claims 89-90, wherein the one or more luminescences and/or determinations of light signals at the excitation wavelength are measured polarization-selective, wherein preferably the one or more luminescences are measured at a polarization that is different from the one of the excitation light.

93. A method according to any of claims 81-92, comprising the determination of changes in the effective refractive index on the measurement areas in addition to determining the one or more luminescences.

94. A method according to any of claims 59-93, comprising the density of the recognition elements immobilized in discrete measurement areas for the detection of different analytes on different measurement areas being selected in such a way that the signals upon determination of different analytes in a common array are of similar order of magnitude, i.e. that the related calibration curves for the analyte determinations to be performed at the same time can be recorded without a change in the settings of the electronic or opto-electronic system.

95. A method according to any of claims 59-94, comprising arrays of measurement areas being divided into segments of one or more measurement areas for the determination of analytes and regions between these measurement areas or additional measurement areas for the purpose of the physical referencing, for example, of the excitation light intensity available in the measurement areas or of the influence of changes in external parameters, such as temperature, and for the purpose of referencing the influence of additional physicochemical parameters, such as nonspecific binding of components of an applied sample to the sensor platform.

96. A method according to any of claims 59-95, comprising one or more measurement areas of a segment or an array being assigned to the determination of the same analyte and the immobilized biological or biochemical or synthetic recognition elements thereof having differing degrees of affinity to said analyte

97. A method according to any of claims 72-96, comprising additional arrangements being made for locally resolved referencing of the excitation light intensity available in the measurement areas.

98. A method according to claim 97, comprising the locally resolved referencing of the excitation light intensity available in the measurement areas including the simultaneous or sequential generation of an image of the light emanating from the sensor platform at the excitation wavelength.

99. A method according to any of claims 97-98, comprising the locally resolved referencing of the excitation light intensity available in the measurement areas being performed by means of “luminescence marker spots”, i.e. determination of luminescence intensity from measurement areas with pre-immobilized luminescently labeled molecules (i.e. molecules which have already been deposited in these measurement areas before application of a sample).

100. A method according to any of claims 59-99, comprising one or more samples being incubated beforehand with a mixture of the various detection reagents for determining the analytes to be detected in said samples and these mixtures then being added in a single step to the related dedicated arrays on the sensor platform.

101. A method according to any of claims 59-100, comprising the concentration of the detection reagents, such as secondary tracer antibodies and/or labels and optional additional labeled tracer reagents in a sandwich immunoassay, being selected in such a way that the signals upon the detection of different analytes in a common array are of the same order of magnitude, i.e. that the related calibration curves for the analyte determinations to be carried out simultaneously can be measured without a change in the settings of the electronic or opto-electronic system.

102. A method according to any of claims 72-101, wherein the calibration of luminescences generated as a result of the binding of one or more analytes or resulting from the specific interaction with one or more analytes comprises the application of one or more calibration solutions with known concentrations of said analytes to be determined to the same or different measurement areas or segments of measurement areas or arrays of measurement areas on a sensor platform to which one or more of the samples to be tested are added in the same or in a separate step.

103. A method according to any of claims 72-102, wherein the calibration of luminescences generated as a result of the binding of one or more analytes or resulting from specific interaction with one or more analytes comprises the comparison of the luminescence intensities after addition of an unknown sample and a control sample, such as a “wild type” DNA sample and a “mutant DNA” sample.

104. A method according to claim 103, comprising the application of unknown sample and control sample to different arrays.

105. A method according to claim 103, comprising the application of unknown sample and control sample sequentially to the same array.

106. A method according to claim 103, comprising the unknown sample and the control sample being mixed and the mixture then being applied to one or more arrays of a sensor platform.

107. A method according to any of claims 103-106, comprising the detection of the analytes to be determined in the unknown and the control sample being carried out using luminescence labels of different excitation and/or luminescence wavelengths for the unknown and the control sample.

108. A method according to any of claims 72-107, comprising several measurement areas with biological or biochemical or synthetic recognition elements immobilized there in differing controlled density being provided in one or more arrays for the determination of an analyte common to these measurement areas.

109. A method according to claim 108, comprising the possibility of establishing a calibration curve for an analyte with the application of just a single calibration solution when the concentration dependence of the binding signals between the analyte and its biological or biochemical or synthetic recognition elements is known and there is a sufficiently wide “variation” of these recognition elements immobilized in different controlled density in different measurement areas of an array.

110. A method according to any of claims 59-109 for simultaneous or sequential, quantitative or qualitative determination of one or more analytes from the group of antibodies or antigens, receptors or ligands, chelators or “histidine tag components”, oligonucleotides, DNA or RNA strands, DNA or RNA analogs, enzymes, enzyme cofactors or inhibitors, lectins and carbohydrates.

111. A method according to any of claims 59-110, comprising the samples to be tested being naturally occurring body fluids such as blood, serum, plasma, lymph or urine or egg yolk or optically turbid fluids or tissue fluids or surface water or soil or plant extracts or biological or synthetic process broths or being taken from biological tissue parts or from cell cultures or extracts.

112. The use of a kit according to any of claims 1-57 and/or a method according to one of claims 59-11 for quantitative or qualitative analysis for the determination of chemical, biochemical or biological analytes in screening methods in pharmaceutical research, combinatorial chemistry, clinical and preclinical development, for real-time binding studies and for the determination of kinetic parameters in affinity screening and in research, for qualitative and quantitative analyte determinations, especially for DNA- and RNA analysis, for the generation of toxicity studies and for the determination of gene and protein expression profiles, and for the determination of antibodies, antigens, pathogens or bacteria in pharmaceutical product development and research, human and veterinary diagnostics, agrochemical product development and research, for symptomatic and presymptomatic plant diagnostics, for patient stratification in pharmaceutical product development and for therapeutic drug selection, for the determination of pathogens, noxious substances and pathogens, especially salmonella, prions and bacteria, in food and environmental analysis.
